(19) United States
(12) Patent Application Publication
(10) Pub. No.: US 2026/0273343 A1
(43) Pub. Date: Sep. 17, 2026

(54) PHYSICAL THERAPY AND FITNESS SYSTEM AND METHOD

(71) Applicant: PLAYTIME LLC, Holland, OH (US)

(72) Inventor: Caroline Humberston, Holland, OH (US)

(21) Appl. No.: 19/566,362

(22) Filed: Mar. 13, 2026

Related U.S. Application Data
(60) Provisional application No. 63/771,172, filed on Mar. 13, 2025.

(51) Int. Cl. A63B 24/00; A63B 71/06; G16H 15/00

(52) U.S. Cl. CPC A63B 24/0075; A63B 71/0622; G16H 15/00; A63B 2024/0068; A63B 2220/05; A63B 2220/806

(57) ABSTRACT

Ways for clinical assessment of a physical movement of a user and progressive improvement of the physical movement guided by an instructor are provided. A system can include a memory having a database and processor-executable instructions including an image recognition model, a large language model, a tracing engine, an assessment and education platform, and a library. The image recognition model can analyze a video recording and generate a suggested score. An instructor interface can receive the suggested score, and an instructor can accept or reject the suggested score to provide final grading data. The large language model can generate a summary based on the final grading data. The tracing engine can overlay real-time visual indicators on video recordings with color-coded feedback. An education module can track lesson completion and automatically prompt the user to submit another video. The system can generate movement improvement metrics by comparing assessments across time points.

### CROSS-REFERENCE TO RELATED APPLICATIONS

This application claims the benefit of U.S. Provisional Application No. 63/771,172, filed on March 13, 2025. The entire disclosure of the above application is incorporated herein by reference.

### FIELD

The present technology relates to a system and method for physical therapy and fitness assessment and education, and, more particularly, to a system and method for clinical assessment of a physical movement of a user and progressive improvement of the physical movement guided by an instructor for enhancing proprioceptive senses and teaching movement literacy.

### INTRODUCTION

This section provides background information related to the present disclosure which is not necessarily prior art.

Individuals of all ages may experience reduced physical activity due to sedentary lifestyles, including spending less time outdoors and less time engaging in physical activities. Increased reliance on technology, prolonged sitting during work or leisure, and decreased engagement in routine physical activities may contribute to a rise in health issues, including injuries and premature physical conditions such as joint stiffness, muscle atrophy, arthritis, increased incidence of ACL tears, and poor posture. These conditions may lead to chronic pain, mobility limitations, and an overall decline in physical well-being.

Decreased physical activity may also lead to a decrease in sensory perception, which in turn may place individuals at a disadvantage for intuiting movements, postures, balance, or engaging muscle groups. Additionally, when a majority of the physical activities of an individual center around an organized sport, the physical movements may be repetitive and may not provide a wide range of motions and physical exercises. Over time, the lack of variation in physical activity may lead to an individual prematurely physically wearing down. Individuals may further lack knowledge on specific indicators such as pain or discomfort when conducting activities improperly, compounding the risk of early onset medical conditions.

While some individuals attempt to counteract a sedentary habit through participation in organized sports, these activities often focus on specific skills rather than comprehensive physical health. Many sports emphasize repetitive movements within a limited range of motion, potentially neglecting aspects of overall fitness such as flexibility, balance, and stability. Combined with the body’s physical changes during adolescent growth, a sedentary lifestyle or repetitive movements can contribute to movement dysfunctions that may later contribute to injuries, pain issues, joint wear, or muscle atrophy. As a result, the individual may not achieve the full benefits needed for long-term physical health and avoiding injuries. While coaches and parents may assist individuals in physical activities and physical exercises, the exercises that coaches may advise individuals to engage in can be sports specific and may not include movements that provide success and physical longevity on a clinical level.

Certain web-based and application-based systems focus on fitness or measure specific parameters like range of motion or strength. Various health and fitness applications may promote active lifestyles; however, these applications may primarily focus on general fitness tracking, step counting, or guided workouts. Certain health and fitness applications use video to track performance or analyze movement, but this is often of a group of individuals or a very detailed synopsis of movement pertinent to specialty applications like a specific sport or rehabilitation. Popular fitness applications fall into several categories, including activity trackers, workout training applications, coaching analysis applications, yoga and flexibility applications, and rehabilitation applications. Activity trackers may monitor steps, heart rate, and daily movement but may not provide guidance on proper movement mechanics or injury avoidance. Workout and training applications may offer structured workouts, but they primarily target cardiovascular health and strength training rather than addressing flexibility, strength, stability, and joint health, yet do not measure movement. In turn, they rely on self-assessment. Yoga and flexibility applications may provide guided stretching and yoga routines, yet these applications often lack a clinical perspective on mobility and balance improvement. Rehabilitation applications may focus on specific rehabilitation exercises for injuries but may not be configured for comprehensive and longevity-focused physical activity or physical literacy.

Despite the wide range of fitness applications available, certain other applications may lack the clinical insights necessary to address foundational aspects of movement, including range of motion, balance, stability, and flexibility. Certain applications are stand-alone applications and do not include in-person training programs, books, or visual aids to assist in the learning process, targeting all learning styles. Without a structured, clinical approach to movement education and exercise, a user may remain at risk of developing musculoskeletal imbalances and other physical health concerns. Certain other fitness applications may not encourage and teach body awareness and may lack the features necessary to educate the user on balancing movements to avoid unnecessary long-term pain. Therefore, users of certain fitness applications may not learn about how their body should move and how to ensure that their body can be moving in the intended way.

Accordingly, there is a continuing need for a web-based or application-based system configured to assist individuals in improving physical activity levels through clinically guided movements and educational resources. Desirably, such a system would provide personalized guidance to enhance overall physical longevity and instruct physical literacy, movement literacy, and body awareness by incorporating techniques that promote functional movement, joint mobility, and stability. Desirably, such a system would integrate clinical expertise with technology to help the user maintain long-term physical health and improve overall well-being while addressing the limitations found in other fitness applications that do not incorporate clinical perspectives on movement mechanics, body awareness, and injury prevention. Desirably, such a system would enable quantitative tracking of movement improvement over time through iterative assessments and automated progress monitoring, providing measurable feedback that other fitness applications cannot achieve.

### SUMMARY

In concordance with the instant disclosure, a system and method for clinical assessment of a physical movement of a user and progressive improvement of the physical movement guided by an instructor for enhancing proprioceptive senses have surprisingly been discovered. The present technology includes articles of manufacture, systems, and processes that relate to assessing and educating individuals and groups on physical movements for enhancing proprioceptive senses and promoting physical longevity through clinically guided instruction.

In certain embodiments, a system for clinical assessment of a physical movement of a user and progressive improvement of the physical movement guided by an instructor can be provided. The system can include a memory having a database and processor-executable instructions including an image recognition model, a large language model, a tracing engine, an assessment and education platform, and a library. The image recognition model can be trained on the database and can be configured to receive a video recording depicting the physical movement of the user, analyze the video recording to identify a movement characteristic, and generate a suggested score based on the identified movement characteristic. The large language model can be trained on the database and can be configured to receive grading data, analyze the grading data by referring to the database, and generate a summary based on the analyzed grading data. The tracing engine can overlay a visual indicator on the video recording in real-time as the video recording can be captured, track performance of the user during the physical movement, display a first color indicator when the physical movement can be aligned with a correct positioning, and display a second color indicator when the physical movement can require adjustment. The assessment and education platform can include an input module configured to receive user identification data and a plurality of video recordings depicting the physical movement captured at different time points. The assessment and education platform can include an assessment module configured to provide the suggested score from the image recognition model to an instructor interface, receive instructor input accepting or rejecting the suggested score, receive instructor comments when the suggested score can be rejected, receive final grading data from the instructor interface based on the instructor input, provide the final grading data to the large language model, receive the summary from the large language model, compare assessments across the different time points to generate a movement improvement metric, generate score comparison data enabling comparison of scores across the different time points, store assessments as the historical assessment data, and enable sharing of the final grading data and the movement improvement metric to an external healthcare provider. The assessment and education platform can include an education module configured to receive a plurality of lessons selected by the instructor interface, receive importance rankings for the plurality of lessons from the instructor interface, order the plurality of lessons in a prioritized sequence based on the importance rankings, display the final grading data, the summary, and the plurality of lessons in the prioritized sequence to the user, maintain an activity log tracking completion status of each lesson, automatically prompt the user to submit a subsequent video recording when a condition selected from a group consisting of completion of a predetermined number of lessons and elapse of a predetermined time period can be satisfied, display the score comparison data and the movement improvement metrics to the user, enable the user to download the final grading data, the movement improvement metrics, and the historical assessment data as a local record, and enable the user to schedule an appointment with an instructor through the instructor interface. The library can contain a plurality of library items organized into categories, and the plurality of lessons can include library items selected from the library based on movement deficiencies identified in the final grading data.

In certain embodiments, a method for clinical assessment of a physical movement of a user and progressive improvement of the physical movement guided by an instructor can be provided. The method can include providing a system including a memory having a database and processor-executable instructions including an image recognition model, a large language model, a tracing engine, an assessment and education platform, and a library, and a processor configured to access the memory and execute the processor-executable instructions. The method can include receiving, via an input module, a first video recording depicting the physical movement of the user at a first time point. The method can include overlaying, via the tracing engine, a visual indicator on the first video recording as the first video recording can be captured, wherein the visual indicator can display a first color when the physical movement can be aligned with a correct positioning and a second color when the physical movement can require adjustment. The method can include analyzing, via the image recognition model, the first video recording to generate a first suggested score. The method can include providing the first suggested score to an instructor interface. The method can include receiving, from the instructor interface, instructor input accepting or rejecting the first suggested score. The method can include, when the first suggested score can be rejected, receiving instructor comments from the instructor interface. The method can include receiving, from the instructor interface, a first final grading data based on the instructor input. The method can include analyzing, via the large language model, the first final grading data to generate a first summary. The method can include receiving, from the instructor interface, a plurality of lessons and importance rankings for each lesson. The method can include ordering the plurality of lessons in a prioritized sequence based on the importance rankings. The method can include displaying, to the user via an education module, the first final grading data, the first summary, and the plurality of lessons in the prioritized sequence. The method can include tracking completion of lessons in an activity log. The method can include automatically prompting the user to submit a second video recording when a condition selected from a group consisting of completion of a predetermined number of lessons and elapse of a predetermined time period can be satisfied. The method can include receiving, via the input module, the second video recording depicting the physical movement at a second time point. The method can include analyzing, via the image recognition model, the second video recording to generate a second suggested score. The method can include receiving, from the instructor interface, second final grading data. The method can include comparing the first final grading data to the second final grading data to generate a movement improvement metric. The method can include generating score comparison data comparing the first final grading data and the second final grading data. The method can include displaying, to the user, the movement improvement metric and the score comparison data. The method can include enabling the user to share the first final grading data, the second final grading data, and the movement improvement metric to an external healthcare provider. The method can include enabling the user to download the first final grading data, the second final grading data, the movement improvement metric, and historical assessment data as a local record. The method can include enabling the user to schedule an appointment with the instructor through the instructor interface.

In certain embodiments, a non-transitory computer-readable medium storing processor-executable instructions can be provided. When executed by a processor, the instructions can cause the processor to perform a method for clinical assessment of a physical movement of a user and progressive improvement of the physical movement guided by an instructor. The method can include receiving a first video recording depicting the physical movement of the user at a first time point. The method can include overlaying a visual indicator on the first video recording in real-time as the first video recording can be captured, wherein the visual indicator can display a first color when the physical movement can be aligned with a correct positioning and a second color when the physical movement can require adjustment. The method can include analyzing, via an image recognition model trained on a database, the first video recording to generate a first suggested score. The method can include providing the first suggested score to an instructor interface. The method can include receiving instructor input accepting or rejecting the first suggested score. The method can include, when the first suggested score can be rejected, receiving instructor comments from the instructor interface. The method can include receiving first final grading data from the instructor interface based on the instructor input. The method can include analyzing, via a large language model trained on the database, the first final grading data to generate a first summary. The method can include receiving a plurality of lessons and importance rankings for each lesson from the instructor interface. The method can include ordering the plurality of lessons in a prioritized sequence based on the importance rankings. The method can include displaying the first final grading data, the first summary, and the plurality of lessons in the prioritized sequence to the user. The method can include tracking completion of lessons in an activity log. The method can include automatically prompting the user to submit a second video recording when a condition selected from a group consisting of completion of a predetermined number of lessons and elapse of a predetermined time period can be satisfied. The method can include receiving the second video recording depicting the physical movement at a second time point. The method can include analyzing, via the image recognition model, the second video recording to generate a second suggested score. The method can include receiving second final grading data from the instructor interface. The method can include comparing the first final grading data to the second final grading data to generate a movement improvement metric. The method can include generating score comparison data comparing the first final grading data and the second final grading data. The method can include displaying the movement improvement metric and the score comparison data to the user. The method can include enabling the user to share the first final grading data, the second final grading data, and the movement improvement metric to an external healthcare provider. The method can include enabling the user to download the first final grading data, the second final grading data, the movement improvement metric, and historical assessment data as a local record. The method can include enabling the user to schedule an appointment with the instructor through the instructor interface.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a block diagram illustrating a system for clinical assessment of a physical movement of a user and progressive improvement of the physical movement guided by an instructor, according to an embodiment of the present disclosure;

FIG. 2, is a block diagram illustrating a database of the system, according to the embodiment shown in FIG. 1;

FIG. 3 is a is a block diagram illustrating an assessment and education platform of the system, according to the embodiment shown in FIG. 1;

FIG. 4 is a is a block diagram illustrating tangible, non-transitory, processor-executable instructions of the system, according to the embodiment shown in FIG. 1;

FIG. 5A and 5B illustrate a physical movement of a user, according to an embodiment of the present disclosure; and

FIGS. 6-10 provide a flow chart illustrating a method for clinical assessment of a physical movement of a user and progressive improvement of the physical movement guided by an instructor, according to an embodiment of the present disclosure.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1–10, or 2–9, or 3–8, it is also envisioned that Parameter X may have other ranges of values including 1–9, 1–8, 1–3, 1–2, 2–10, 2–8, 2–3, 3–10, 3–9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature’s relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The present technology can provide a system that helps people improve their physical movements through a combination of video analysis, artificial intelligence (AI), and expert instruction. A user can record themselves performing specific physical movements such as squats, lunges, pushups, or any other physical movement, and upload these video recordings to the system. An artificial intelligence system can analyze the videos and suggest scores based on factors like posture, balance, flexibility, and proper form. An instructor, such as a physical therapist or coach, can then review both the video and the AI suggestions, accepting or modifying the scores and providing personalized feedback. Another AI component can generate a detailed summary explaining what the user did well and what needs improvement, along with customized lessons to address specific deficiencies.

One aspect of the present technology can include a real-time tracing engine that provides visual feedback while the user may be performing movements. As the user executes a movement, the system can overlay one or more colored indicators on a live video feed, for example, showing green when positioning is correct and showing red when adjustment may be needed. This allows the user to make corrections in real-time rather than discovering mistakes after the fact. The system can also include an activity tracker that monitors which educational lessons the user completes and automatically prompts them to submit new videos at appropriate intervals, ensuring consistent progress tracking.

The present technology can excel at tracking improvement over time by comparing assessments from different dates or times and generating visual progress reports. The user can see exactly how their movement quality has improved, which specific areas got better, and which deficiencies persist. For group settings like physical education classes or sports teams, the system can analyze multiple users simultaneously, identify common problems across the group, and recommend lessons. The one or more users can share their progress with healthcare providers, download their records, and even schedule one-on-one appointments with instructors for personalized guidance.

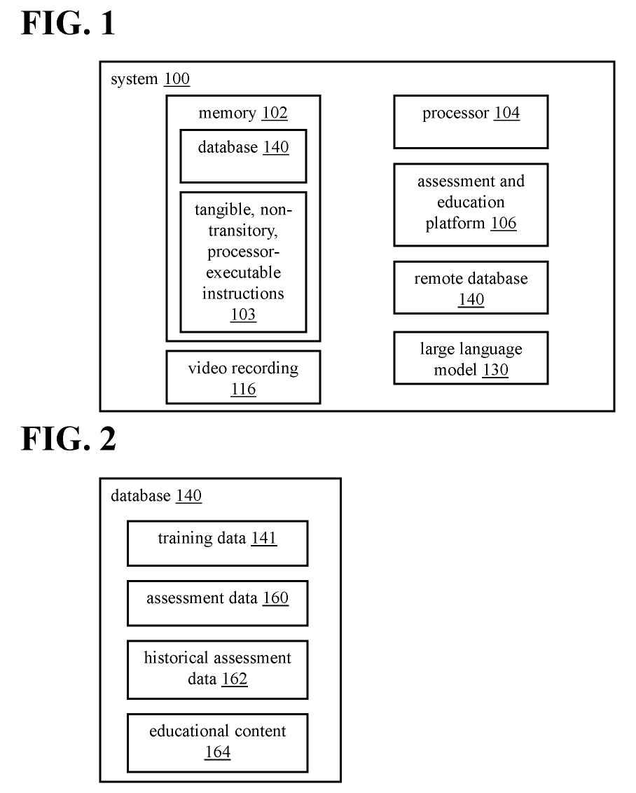

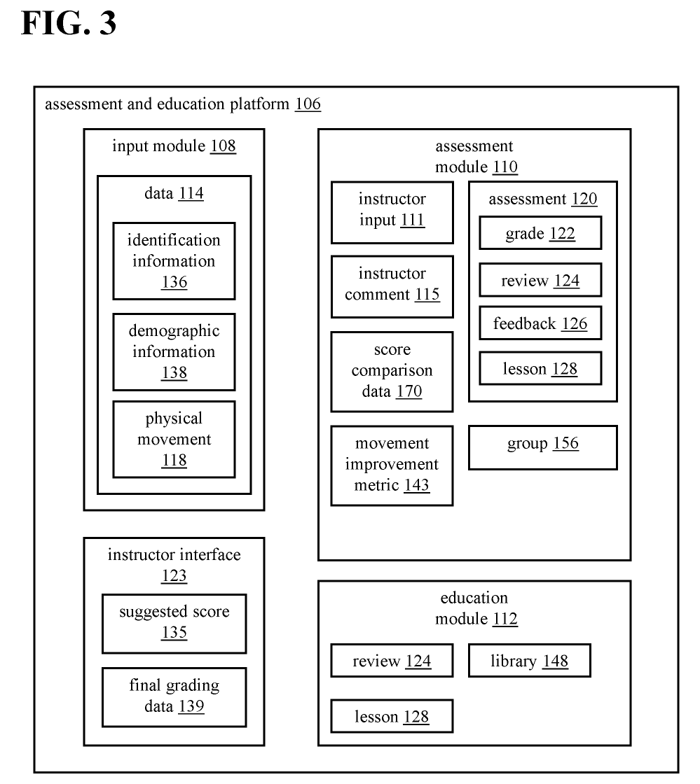

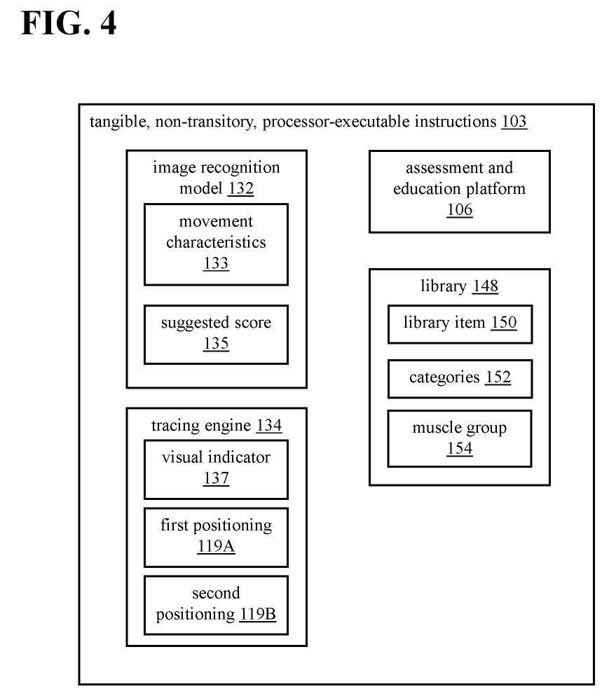

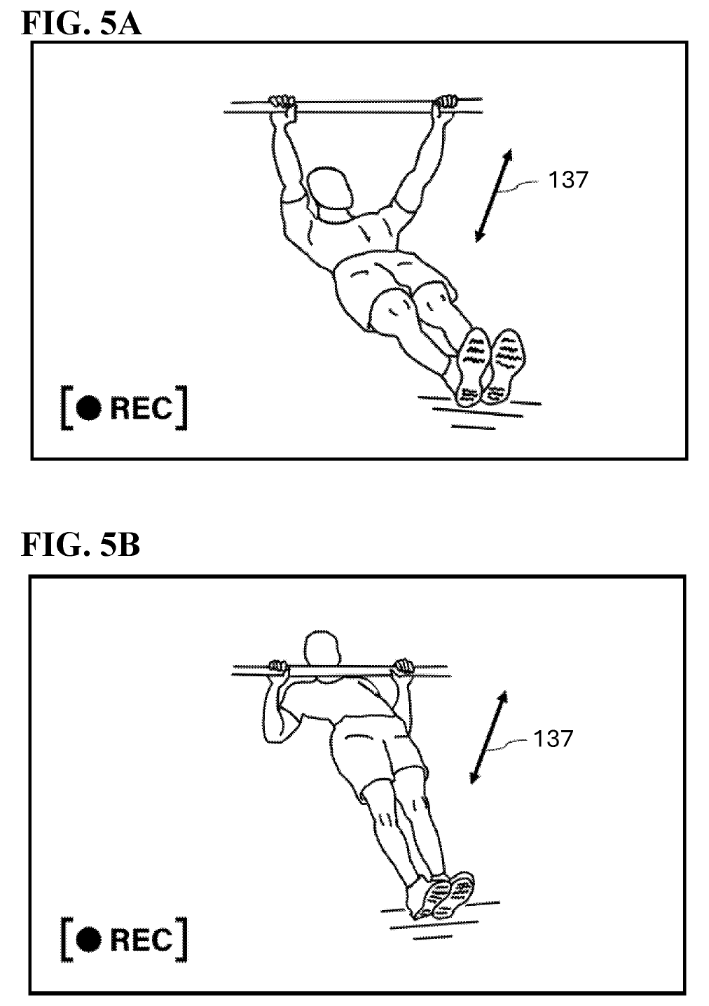

As shown in FIGS. 1-5B, a system 100 for assessment and education of physical activities for a user by an instructor can be provided. The system 100 can bridge the gap between the clinical aspects of fitness and the effectiveness of an interactive platform, allowing the user to provide data 114, for example, the age and gender of the user, and upload a video recording 116 of the user executing a physical movement 118 so that an instructor can assess the balance, stability, flexibility, strength, and posture of the user. The system 100 can allow the instructor to provide an assessment 120 of the physical movement 118. The assessment 120 can include a grade 122, a review 124, feedback 126, and a lesson 128 to assist the user with physical longevity, movement mechanics, and strategies to help militate against pain and injuries. The system 100 may also harness artificial intelligence including a large language model 130, an image recognition model 132, and a tracing engine 134 to supplement the grade 122, review 124, and feedback 126. The user may include a student, an individual seeking clinically based fitness instructions, or a member of a fitness group. The instructor may include a physical therapist, physical education teacher, medical professional, class instructor, or coach. The instructor may also include a group administrator.

The system 100 can include a memory 102, and a processor 104 coupled with the memory 102. The system 100 can include an assessment and education platform 106. The assessment and education platform 106 can include an input module 108, an assessment module 110, and an education module 112. The system 100 can receive data 114 from the user via the input module 108 on the assessment and education platform 106. The data 114 may include identification information 136 and demographic information 138 of a user. It should be appreciated that data 114 may be uploaded through the input module 108 and transferred to the assessment module 110, the education module 112, or to a remote database 140 as needed.

The memory 102 can include a database 140 and tangible, non-transitory, processor-executable instructions 103. The database 140 can store training data 141, assessment data 160, historical assessment data 162, and educational content 164. The tangible, non-transitory, processor-executable instructions 103 can include an image recognition model 132, a large language model 130, a tracing engine 134, the assessment and education platform 106, and a library 148.

The processor 104 can be configured to access the memory 102 (including the database 140) and execute the tangible, non-transitory, processor-executable instructions. The processor 104 may include a central processing unit (CPU), a microprocessor, a microcontroller, or a system-on-a-chip. The processor 104 may include a single processor or multiple processors in a single processing unit or multiple processing units and may include multiple processors where one processor can be capable of executing one or more of the elements described herein, and a subsequent processor or processors may execute other elements as described herein.

The memory 102 may include a single memory or multi-memory unit. The memory 102 can store data 114 uploaded from the user, including identification information 136 such as name and ID number, and demographic information 138 such as age and gender. The memory 102 may also store video recordings 116 of the user. The database 140 within the memory 102 can be stored locally on a computer of the user or instructor, or on a remote server, depending on the needs of the user, instructor, or organization utilizing the assessment and education platform 106.

The input module 108 can allow the user to provide data 114 to the assessment and education platform 106 in order for the system 100 to assess the user. The data 114 may include identification information 136 and demographic information 138 of the user such as the name, age, and gender of the user, and may also include an ID number in order to anonymize the user on the assessment and education platform 106. The data 114 may include one or more movements 118. The movements 118 may include, for example, a bodyweight squat, a forward lunge, a hinge, a standing rotation, a modified pushup, a modified plank, and a modified pullup. The input module 108 can be configured to receive user identification data and a plurality of video recordings 116 depicting the physical movement 118 captured at different time points. The video recordings 116 may be captured using a camera associated with a computer, which may include a desktop computer, a laptop computer, a tablet device, and a mobile smartphone. The video recordings 116 can depict the user performing the physical movement 118, and can be uploaded to the input module 108 for analysis.

The image recognition model 132 can be trained on the database 140 and can receive a video recording 116 depicting the physical movement 118 of the user. The image recognition model 132 can analyze the video recording 116 to identify one or more movement characteristics 133 and generate a suggested score 135 based on the identified movement characteristic(s) 133. The image recognition model 132 can employ computer vision techniques to analyze the video recording 116 and identify physical characteristics of the physical movement 118. The identified movement characteristic 133 from the image recognition model 132 can include posture, spinal alignment, body angle, foot positioning and ankle flexibility, knee tracking, hip positioning, shoulder positioning, balance, stability, flexibility, range of motion, joint angles, movement symmetry, and center of mass displacement. The image recognition model 132 can analyze the identified movement characteristic 133 and generate a suggested score 135 that may include a numerical score, a binary completion indicator, individual scores for multiple movement criteria, or combinations thereof. In certain embodiments, the image recognition model 132 can be configured to analyze the video recording 116 without access to user identifying information to protect user privacy. The video recording 116 provided to the image recognition model 132 may be strategically altered or processed to obscure identifying features such as facial features, tattoos, birthmarks, scars, and distinctive physical markings. This processing can protect the privacy and anonymity of the user while still allowing the image recognition model 132 to analyze movement patterns.

The database 140 can include annotated training data 141 including previously graded video recordings with corresponding movement deficiency labels. Both the image recognition model 132 and the large language model 130 can be trained on this annotated training data 141. The training data 141 can allow the models to learn correlations between visual patterns in video recordings and movement quality assessments, enabling accurate analysis of new video recordings

The image recognition model 132 can employ techniques such as pose estimation algorithms to identify anatomical keypoints in each frame of the video recording 116. The anatomical keypoints may include spine, head, shoulders, elbows, wrists, hips, knees, and ankles. The image recognition model 132 can calculate joint angles between body segments using the identified keypoints, and can compare these measurements to clinical reference ranges stored in the database 140 to generate a quantitative movement quality scores. The quantitative movement quality scores can be compiled by the image recognition model 132 into a suggested score 135 which can be transmitted to the assessment module 110 and provided to the instructor via the instructor interface for review, validation, and acceptance or rejection as part of the assessment 120 workflow.

The large language model 130 can be trained on the database 140 and can be configured to receive grading data, analyze the grading data by referring to the database 140, and generate a summary based on the analyzed grading data. The large language model 130 can review submitted content and refer to the database 140 to compose a response 146. The large language model 130 can receive grading data from the assessment module 110 after an instructor has reviewed and validated the suggested score 135 from the image recognition model 132. The grading data can include the final score assigned by the instructor, any modifications made to the suggested score, and instructor comments explaining the grading decision. The large language model 130 can analyze this grading data in the context of information stored in the database 140, including clinical knowledge about movement mechanics, common movement deficiencies, and appropriate corrective interventions. Based on this analysis, the large language model 130 can generate a summary that explains the assessment 120 results to the user in clear, understandable language. The summary can describe what the user performed correctly, identify specific areas needing improvement, explain why certain movement patterns may be problematic, and provide context for the assigned lessons 128. The summary can incorporate instructor comments and can align with the instructor’s input, ensuring that the automated summary reinforces the clinical guidance provided by the instructor.

The large language model 130 can be trained on a corpus of previously graded assessments 120 with corresponding expert-written summaries, allowing it to learn appropriate language, tone, and content structure for communicating assessment 120 results. The training process can enable the large language model 130 to generate summaries that can be clinically accurate, pedagogically effective, and appropriately personalized to the user’s specific movement deficiencies.

The tracing engine 134 can be configured to overlay a visual indicator 137 on the video recording 116 in real-time as the video recording 116 can be captured, track performance of the user during the physical movement 118, display a first color indicator when the physical movement 118 can be aligned with a first positioning 119A that is correct, and display a second color indicator when the physical movement 118 can be aligned with a second positioning 119B that is incorrect. The tracing engine 134 can provide real-time visual feedback as the user performs physical movements 118. Unlike post-processing analysis that provides feedback 126 after a movement can be complete, the tracing engine 134 can overlay the visual indicator 137 on a live video feed as the movement can be executed, enabling immediate correction and learning. The tracing engine 134 can be implemented using computer vision techniques to track the user’s body position in real-time. As the user performs a physical movement 118, the tracing engine 134 may identify anatomical keypoints such as head, spine, shoulders, hips, knees, and ankles in each video frame. The tracing engine 134 can calculate the positions of these keypoints relative to reference positions stored in the database 140 that define correct and incorrect positioning for the specific movement 118.

The tracing engine 134 can overlay the visual indicator 137 on the live video feed to show the user whether their positioning can be correct. The visual indicator 137 may include one or more dots positioned at anatomical keypoints, one or more lines connecting keypoints to show body segment alignment, one or more arrows indicating direction of correction, one or more reference alignment markers showing ideal positioning, and combinations thereof. The visual indicators 137 can be color-coded to provide immediate, intuitive feedback 126. In certain embodiments, the first color indicator can be green and the second color indicator can be red. The tracing engine 134 can display a green visual indicator 137 when the user’s positioning can be aligned with correct form, and a red visual indicator 137 when the user’s positioning can require adjustment. For example, during a forward lunge, the tracing engine 134 may overlay a green line along the user’s front shin when the knee can be properly positioned over the ankle, and a red line when the knee tracks too far forward.

The tracing engine 134 can be guided by analysis from the image recognition model 132 to determine whether the physical movement 118 can be aligned with correct positioning. The image recognition model 132 can provide real-time analysis of movement quality to inform the tracing engine 134 determination of correct versus incorrect positioning. The tracing engine 134 can track performance of the user during the physical movement 118 by identifying an anatomical keypoint in each frame of the video recording 116, calculating a position of the anatomical keypoint relative to a reference position stored in the database 140, and determining alignment based on whether the calculated position can be within a predetermined tolerance of the reference position. When the calculated position can be within the predetermined tolerance, the tracing engine 134 can display the first color indicator (e.g., green), and when the calculated position can be outside the predetermined tolerance, the tracing engine 134 can display the second color indicator (e.g., red).

The real-time nature of the tracing engine 134 can provide a technological advantage over post-processing systems. The user can receive feedback 126 during movement execution, enabling them to make corrections in real-time rather than completing an incorrect movement and only learning about the error afterward. This feedback 126 can accelerate learning and help the user develop proper movement patterns more quickly.

The assessment module 110 can provide the instructor with data 114 and the video recording 116 from the user in order to provide the user with an assessment 120. The assessment 120 may require a video recording 116 of each movement 118 separately. The assessment 120 can score each movement 118 on various characteristics. For example, the movement 118 may be evaluated on posture, whether the user maintains a straight spine, whether the user maintains a correct angle of the body, the feet positioning of the user, or the like.

The assessment module 110 can provide the suggested score 135 from the image recognition model 132 to an instructor interface 123. The instructor interface can be a component of the assessment and education platform 106 that can allow an instructor to interact with the system 100, review assessment 120 data, and provide clinical guidance. The instructor interface can display the suggested score 135 along with the video recording 116, allowing the instructor to review both the AI analysis and the actual movement execution. The assessment module 110 can receive instructor input 111 accepting or rejecting the suggested score. The instructor can accept the suggested score 135 without modification if the AI analysis is accurate, reject the suggested score 135 if the AI analysis is incorrect, or modify the suggested score 135 to adjust specific criteria. This human-in-the-loop validation can ensure clinical accuracy while leveraging the efficiency of automated analysis. The assessment module 110 can receive an instructor comment 115 when the suggested score 135 is rejected. When the instructor rejects or modifies the suggested score, the instructor interface can prompt the instructor to provide the instructor comment explaining the rejection or modification. The instructor comment can provide valuable context for understanding why the AI suggestion was incorrect and can be used to continuously improve the image recognition model 132. The AI assisted analysis can provide busy instructors with a starting point for clinical analysis and provide a way for users to be involved in the education process.

The assessment module 110 can receive a final grading data 139 from the instructor interface based on the instructor input 111. The final grading data 139 can represent the instructor’s validated assessment 120 of the user’s movement quality, incorporating the instructor’s clinical expertise and judgment. The final grading data 139 can include numerical scores for various movement criteria, overall movement quality ratings, identification of specific movement deficiencies, and qualitative observations about the user’s performance. The assessment module 110 can provide the final grading data 139 to the large language model 130. Once the instructor provides final grading data 139, the system 100 can transmit this data to the large language model 130 for generation of a summary. The assessment module 110 can receive the summary from the large language model 130. After the large language model 130 analyzes the final grading data 139 and generates a summary, the assessment module 110 can receive this summary and make it available for display to the user. The summary can be sent to health care providers or downloaded locally.

The assessment module 110 can compare two or more assessments 120 across the different time points to generate a movement improvement metric 143. The movement improvement metric 143 can quantify the user’s progress over time by comparing movement quality scores from different assessments 120. The movement improvement metric 143 can include change in overall movement quality score, change in specific movement criteria scores, percentage improvement calculation, rate of improvement over time, comparison to population benchmarks, and combinations thereof.

The assessment module 110 can generate score comparison data 170 enabling comparison of scores across the different time points. The score comparison data 170 can include a side-by-side numerical comparison of scores from assessments 120 at different time points, visual highlighting of improved movement criteria, visual highlighting of persistent movement deficiencies, and a net improvement calculation. The score comparison data 170 can allow the user and instructor to quickly understand which aspects of movement have improved and which areas continue to need attention. The assessment module 110 can store assessments 120 as historical assessment data. Each assessment 120 can be stored in the database 140 as part of the user’s historical assessment data, creating a record of the user’s movement quality over time. This historical data can enable trend analysis, progress tracking, and identification of long-term patterns. The assessment module 110 can enable sharing of the final grading data 139 and the movement improvement metric 143 to an external healthcare provider. The user can share their assessment 120 results and progress data with healthcare providers such as physicians, physical therapists, or sports medicine specialists. This sharing capability can facilitate integration of the system 100 with clinical care and can allow healthcare providers to monitor patient progress remotely.

In certain embodiments, enabling sharing of the final grading data 139 and the movement improvement metrics 143 to an external healthcare provider can include generating a shareable report containing the final grading data 139, the movement improvement metrics 143, and the historical assessment data, encrypting the shareable report, and transmitting the encrypted shareable report to a recipient designated by the user. The shareable report can be formatted for integration with an electronic health record system, facilitating seamless incorporation of movement assessment data into clinical documentation.

In certain embodiments, the education module 112 can include a library 148, a review 124, a feedback 126, and a grade 122 provided by the instructor. The library 148 can be part of the education module 112 on the assessment and education platform 106. Alternatively, the library 148 can be located on the remote server 144, for example, in the database 140. The education module 112 can receive one or more lessons 128 selected by the instructor. After reviewing the assessment 120 and identifying movement deficiencies, the instructor can select an appropriate lesson 128 from the library 148 to address those deficiencies. The instructor can choose the lesson 128 that targets specific movement problems, provides foundational movement education, or advances the user’s skills. It should also be understood that the library 148 can include physical resources such as books, charts, visual aids, guides, instruction manuals, posters, and the like, for example. The education module 112 can direct the instructor and/or the user to such physical resources and include such physical resources in the lessons 128.

The education module 112 can receive importance rankings for the lesson 128 from the instructor interface. The instructor can assign importance rankings to each selected lesson 128 based on factors such as severity of identified movement deficiencies, risk of injury associated with the movement deficiencies, foundational nature of the movement skill, prerequisite relationships between lessons, and user-specific goals. These rankings can guide the sequence in which the user should complete the lesson 128. The education module 112 can order the lessons 128 in a prioritized sequence based on the importance rankings. The education module 112 can automatically sort the selected lessons 128 according to the importance rankings provided by the instructor, creating a structured learning pathway for the user. The prioritized sequence can ensure that the user addresses the most important movement deficiencies first and progresses through lessons 128 in a predetermined order.

The education module 112 can display the final grading data 139, the summary, and the plurality of lessons 128 in the prioritized sequence to the user. Upon receiving the review 124 and lessons 128 from the instructor via the education module 112, the user can review the lessons 128 and grading on the assessment and education platform 106. The user can navigate through lessons 128 all at once or can review the lessons 128 one-by-one, at the personal pace of the user. The education module 112 can display the plurality of lessons 128 in the prioritized sequence with numerical rankings, indicate which lessons can be highest priority, and recommend an order of completion based on the prioritized sequence. This display can provide guidance to the group administrator or individual user about which lessons 128 to complete first and can help the user understand the relative importance of different educational topics. This can allow the user to understand lessons in importance of different educational topics, and the group administrator to apply the lessons tallied as an order of importance for the group, incorporating them into specific programming. For example, a physical education teacher with a class of 30 may have 29 individuals needing lessons on postural alignment. The physical education teacher can choose from the lessons tallied in order of importance to use in the classroom prior to completing a unit on tennis, relating the postural information to the game.

In certain embodiments, the education module 112 can maintain an activity log tracking completion status of each lesson 128. The activity log can record detailed information about user interaction with educational content. For each lesson 128, the activity log can store a timestamp indicating when the lesson 128 was assigned, a timestamp indicating when the lesson 128 was completed, a duration indicating time spent on the lesson 128, and a completion status indicator. A user can mark particular lessons as favorites to allow quick access and return to the lessons found most valuable. These favorite or listed lessons may serve as an exercise list for workout completion.

In certain embodiments, the education module 112 can automatically prompt the user to submit a subsequent video recording 116 when one or more conditions, such as completion of a predetermined number of lessons 128 and/or elapse of a predetermined time period, is satisfied. The system 100 can use the activity log data to determine when to prompt the user for a follow-up assessment 120. The one or more conditions can include completion of a predetermined number of lessons 128, elapse of a predetermined time period, or a combination of both conditions. In certain embodiments, the education module 112 can determine that the predetermined number of lessons 128 are completed by querying the activity log, and automatically generate and display a prompt to the user requesting submission of the subsequent video recording 116. The prompt can include information such as which lessons were completed, how much time has elapsed since a previous assessment 120, expected improvements based on the completed lessons 128, and combinations thereof. A repeated assessment can provide a user and/or instructor with information about how and when to change the attention of training.

The education module 112 can calculate the predetermined time period based on timestamps in the activity log. The predetermined time period can be calculated from a most recent lesson completion timestamp. This approach can ensure that the timing of follow-up assessments 120 can be based on actual user engagement with educational content rather than arbitrary fixed intervals. The education module 112 can display the score comparison data and the movement improvement metrics 143 to the user. The user can access this information to understand their progress and see how their movement quality has changed over time. The display can include numerical data, visual representations such as graphs and charts, and textual explanations of the movement improvement metrics 143.

In certain embodiments, the assessment module 110 can generate a visual representation of the movement improvement metric 143. The visual representation can include a line graph showing a score trend over time, a bar chart comparing scores at different time points, progress indicators showing advancement toward a clinical or coaching benchmark, and combinations thereof. These visual representations can make progress data more accessible and understandable to the user, instructor, teacher, and/or coach.

In certain embodiments, the assessment module 110 can be configured to enable the user to select any two assessments 120 from the historical assessment data and generate score comparison data comparing the selected assessments 120. This can allow the user to compare their current performance to their initial baseline assessment 120, compare assessments 120 across different time intervals, or compare before-and-after assessments 120 surrounding specific training interventions.

In certain embodiments, the education module 112 can be configured to enable the user to download the final grading data 139, the movement improvement metrics 143, and the historical assessment data as a local record. The local record downloadable by the user can include all assessments 120 in the historical assessment data, all scores and grading data, all movement improvement metrics 143, a list of completed lessons 128, and timestamps for all assessments 120 and lesson completions. The local record can be formatted as a file type such as PDF, CSV, JSON, XML, and combinations thereof.

In certain embodiments, the education module 112 can enable the user to schedule an appointment with an instructor through the instructor interface. Enabling the user or group administrator to schedule an appointment with an instructor can include displaying available appointment times from a calendar associated with the instructor, receiving selection of an appointment time from the user, transmitting appointment confirmation to the user and the instructor, and providing secure video conferencing access at the scheduled appointment time. In certain embodiments, the appointment can be conducted at an additional cost to the user beyond a standard subscription fee, and the system 100 can process payment for the appointment. During the appointment, the instructor can have access to the historical assessment data of the user, completed lessons 128, and movement improvement metric 143, allowing the instructor to provide personalized guidance informed by the user’s complete history in the system 100.

The education module 112 can track which lessons 128 in the prioritized sequence have been completed, automatically advance the user to a next highest-priority lesson upon completion of a current lesson, and update the prioritized sequence based on new assessments 120. This automatic progression can guide the user through their educational pathway and can adjust the plan as the user’s needs change based on improvement or identification of new deficiencies.

In certain embodiments, the library 148 can contain library items 150 organized into categories 152. The lessons 128 can include library items 150 selected from the library 148 based on movement deficiencies identified in the final grading data 139. The library 148 can include customized lessons 128, including text and video content for educational purposes and for keeping the user on track with physical exercises. A library item 150 can include information and information sources. Categories 152 can allow a user or an instructor to organize one or more library items 150 for various educational purposes. The categories 152 can include proprioceptive education variables selected from a group consisting of sensory perception identification, movement variation, and movement application. For example, the categories 152 may include “feel it,” “find it,” “vary it,” and “use it,” indicating the proprioceptive education variable that the category 152 fits within. Categories 152 can include specific topics such as posture, flexibility, stability, balance, and the like. Each category 152 can be linked to a muscle group 154. Each library item 150 can be associated with a category 152, and a single library item 150 can be associated with multiple categories 152. It should be appreciated that a library item 150 can therefore fit within multiple categories 152 and a category 152 may also fit within multiple proprioceptive education variables.

In certain embodiments, each lesson 128 can include library items 150 organized into chapters, textual content explaining movement concepts, video content demonstrating proper movement execution, practice exercises, and assessment checkpoints. The lessons 128 in the assessment and education platform 106 can be configured to reference and align with corresponding content found in supplemental educational materials, such as participant manuals and education manuals or visual aids like spine models, to improve learning accessibility and reinforce educational concepts across multiple formats. For example, an assessment 120 can be assigned at least one library item 150, but a lesson 128 can include an unlimited amount of library content, such as categories 152 or library items 150. The library items 150 can be linked to specific movement deficiency patterns, and the instructor interface can automatically suggest library items 150 based on identified movement deficiencies. When an instructor reviews an assessment 120 and identifies specific deficiencies, the system 100 can recommend relevant library items 150 that address those deficiencies, streamlining the process of creating personalized educational plans.

In certain embodiments, the system 100 can implement a closed-loop feedback mechanism. The system 100 can create a closed loop by quantitatively assessing movement quality via video analysis, selecting targeted educational content based on identified deficiencies, tracking completion of educational content, reassessing movement quality via subsequent video analysis, calculating improvement metrics by comparing assessments 120, and adjusting educational content based on measured results. The system 100 can track user progress over time through multiple assessment 120 cycles, enabling measurable improvement in physical movement quality, and ongoing pertinence of learning content. This iterative process can provide a way to quantitatively measure movement improvement and/or automatically adjust educational content based on progress.

After receiving an initial assessment 120 and completing assigned educational content via the education module 112, the user can upload a subsequent video recording 116 demonstrating the same physical movement(s) 118. The assessment module 110 can generate a new assessment 120 and compare it to the prior assessment 120 to determine movement improvement metrics 143. The movement improvement metrics 143 may include quantitative measurements such as changes in flexibility scores, stability scores, balance scores, range of motion measurements, joint angle measurements, or overall movement quality scores. The system 100 can calculate improvement metrics by comparing movement quality scores across multiple assessments 120. For example, if a first assessment 120 identifies limited hip flexibility during a bodyweight squat (e.g., achieving only 85 degrees of hip flexion), and a subsequent assessment 120 after completing hip mobility lessons 128 shows improved hip flexion (e.g., achieving 105 degrees), the system 100 can calculate a 20-degree improvement metric. This quantitative measurement can enable objective tracking of progress.

Based on the movement improvement metrics 143, the education module 112 can automatically adjust the educational content. When improvement metrics indicate progress in a particular area, the system 100 can advance the user to more challenging material. Conversely, when improvement metrics indicate persistent deficiencies, the system 100 can provide additional remedial content targeting those specific deficiencies. The system may recognize the continued movement deficiencies and recommend further evaluation from the instructor. This automated adjustment can create a closed-loop feedback mechanism where assessment 120 data directly informs educational content selection, and subsequent assessments 120 measure the effectiveness of the educational intervention.

The assessment module 110 can identify persistent movement deficiencies by comparing assessments 120 across multiple time points, determine that a movement deficiency is persistent when scores for a particular movement criterion fail to improve beyond a threshold amount across at least two assessments 120, and flag the persistent movement deficiency for instructor review 124. When a persistent deficiency is identified, the education module 112 can automatically recommend alternative lessons 128 targeting the persistent movement deficiencies. The system may recognize the continue movement deficiencies and recommend further evaluation from the instructor.

The education module 112 can correlate changes in the movement improvement metrics 143 with completed lessons 128, identify which lessons 128 can be most effective for specific movement deficiencies based on the correlation, and prioritize lessons 128 for future users with similar movement deficiencies. The correlation can be performed across multiple users, and lesson effectiveness data can be stored in the database 140 for use in training the large language model 130. This correlation analysis can determine which educational content is most effective for specific movement deficiencies. The education module 112 can track which lessons 128 and library items 150 each user completes, and the assessment module 110 can correlate changes in movement improvement metrics 143 with the completed educational content. For example, if multiple users with limited hip flexibility complete a specific hip mobility lesson 128, and subsequent assessments 120 show an average improvement in hip flexion, the system 100 can identify this lesson 128 as highly effective for hip flexibility deficiencies. This correlation data can be stored in the database 140 and used to prioritize content recommendations for future users with similar deficiencies.

In certain embodiments, the system 100 can support group-based assessment 120 and education, enabling the instructor and group administrator to simultaneously work with multiple users. The assessment module 110 can create a group 156 including a plurality of user contacts. The group 156 can be configured to contain data 114 from a user, video recordings 116, assessments 120, grades 122, reviews 124, or lessons 128. The instructor can add or remove users to the group 156 as desired. Creating a group 156 can include receiving a group name from a group administrator, assigning existing user contacts to the group 156 or creating new user contacts, associating video recordings 116, assessments 120, and lessons 128 with the group 156, and maintaining group membership data in the memory 102. This can allow the group administrator to identify and prioritize movement deficits within the group. For example, if the group administrator is a physical education teacher, the physical education teacher can choose to look at the lesson most imperative to the group’s movement deficits as a whole, apply the lesson in the program to the physical education class, then complement the lesson with a game as in the physical education curriculum.

In certain embodiments, the assessment module 110 can receive a video recording 116 from each user in the group 156, generate suggested scores for each user via the image recognition model 132, provide the suggested scores to the instructor interface, receive final grading data 139 for each user from the instructor interface, calculate an average score for the group 156 based on individual final grading data 139 from the plurality of users, identify common movement deficiencies present across multiple users in the group 156, tally lesson assignments across the plurality of users to determine which lessons 128 can be assigned most frequently, and order lessons 128 for the group 156 in a group prioritized sequence based on the tallying, wherein lessons 128 needed by a majority of users in the group 156 can be prioritized higher. The group administrator will be notified of user completion of lessons/assignments, and instructor completion of scoring/assignment allocation.

The assessment module 110 can display the average score, individual scores for each user, and the group prioritized sequence to a group administrator interface, and enable the instructor or the group administrator to assign lessons 128 to the group 156, to subgroups, or to individual users. The group administrator interface can allow instructors and group administrators working with groups to efficiently manage multiple users, identify common patterns across the group 156, and provide both group-wide and individualized instruction. The assessment module 110 can notify the group administrator when individual users complete assigned lessons 128, track group completion rates, and display group progress metrics showing collective improvement across the plurality of users. This tracking can help instructors monitor engagement and ensure that all members of the group 156 can be progressing through their educational content.

The system 100 can be further configured to provide educational sessions to a group administrator teaching application of assessment 120 results to group instruction. These educational sessions can help instructors and group administrators understand how to interpret assessment 120 data at the group level and how to design effective group programming based on collective needs. The system 100 can implement a workflow integrating artificial intelligence analysis with human instructor validation to ensure assessment 120 accuracy while leveraging the efficiency of automated analysis. This two-stage validation process can distinguish the system 100 from fully automated systems that may lack clinical accuracy and from fully manual systems that cannot scale efficiently.

When a user uploads a video recording 116 via the input module 108, the video recording 116 can be automatically transmitted to the image recognition model 132. The image recognition model 132 can analyze the video recording 116 to identify movement characteristics and generate a suggested score. The suggested score 135 may include numerical scores for various movement criteria, binary completion indicators, or overall movement quality assessments.

The suggested score 135 from the image recognition model 132 can be provided to an instructor interface on the assessment module 110. The instructor interface can display the suggested score 135 along with the video recording 116, allowing the instructor to review both the AI analysis and the actual movement execution. The instructor interface can be configured to display the suggested score 135 from the image recognition model 132 with a visual indicator 137 highlighting an area of concern in the video recording 116. The instructor interface can receive input from the instructor to accept the suggested score 135 without modification, reject the suggested score, or modify the suggested score. When the suggested score 135 is rejected or modified, the instructor interface can receive textual comments from the instructor explaining the rejection or modification. The summary generated by the large language model 130 can incorporate the instructor comments and can align the summary with the instructor input.

This AI-assisted workflow can provide several technological advantages. First, the image recognition model 132 can provide rapid initial analysis, reducing the instructor’s time burden by highlighting areas of concern. The analysis by the image recognition model 132 can also identify one or more issues that the instructor cannot measure or ascertain without the use of specialized equipment, thereby performing a function that the instructor cannot complete on their own. Second, the instructor validation can ensure clinical accuracy and can identify and catch edge cases that AI may misinterpret. Third, the large language model 130 can generate personalized feedback 126 that would be time-consuming for an instructor to write manually for each student. Fourth, the instructor comments captured during validation can provide training data 141 to continuously improve the image recognition model 132 over time.

In certain embodiments, the database 140 can be stored on a remote server 144, and the assessment and education platform 106 can access the database 140 via a network connection. The database 140 can include training data 141 for the image recognition model 132 including annotated video recordings, training data 141 for the large language model 130 including graded assessments 120 with corresponding summaries, reference movement data defining correct positioning for each physical movement 118, population benchmark data for comparison purposes, and lesson effectiveness data correlating lessons 128 with movement improvement outcomes.

In certain embodiments, the database 140 can include annotated training data 141 including previously graded video recordings with corresponding movement deficiency labels. Both the image recognition model 132 and the large language model 130 can be trained on the annotated training data 141. The training data 141 can allow the models to learn correlations between visual patterns in video recordings and movement quality assessments, enabling accurate analysis of new video recordings. The training data 141 for the image recognition model 132 can include multiple (e.g., hundreds to thousands) video recordings that have been manually annotated by clinical experts. Each annotated video recording can include labels identifying specific movement deficiencies, numerical scores for movement quality criteria, and markers indicating anatomical keypoints and their positions throughout the movement. The image recognition model 132 can be trained on this annotated data using machine learning techniques such as supervised learning with convolutional neural networks. The training data 141 for the large language model 130 can include graded assessments 120 paired with expert-written summaries explaining the assessment 120 results. This training data 141 can teach the large language model 130 appropriate language, tone, and content structure for communicating assessment 120 results to the user. The large language model 130 can learn to generate summaries that can be clinically accurate, pedagogically effective, functionally relevant to movements of daily life, and appropriately personalized to specific movement deficiencies.

The reference movement data stored in the database 140 can define correct positioning for each physical movement 118. This reference data can include acceptable ranges for joint angles, body segment positions, and other biomechanical parameters at various phases of each movement. The image recognition model 132 and tracing engine 134 can compare observed movement patterns to this reference data to determine movement quality.

The population benchmark data stored in the database 140 can include statistical distributions of movement quality metrics for various demographic groups. This data can allow the system 100 to compare a user’s performance to population norms, providing context for individual assessment 120 results. For example, the system 100 can inform a user that their hip flexibility score can be above average for their age and gender group.

The lesson effectiveness data stored in the database 140 can include correlations between specific lessons 128 and movement improvement outcomes. This data can be continuously updated as the user completes lessons 128 and demonstrate improvement in subsequent assessments 120. The system 100 can use this effectiveness data to prioritize lesson recommendations and optimize educational content selection.

The system 100 can implement privacy protections during AI analysis. Video recordings 116 transmitted to the image recognition model 132 can be processed to obscure user identifying information, such as facial features, ensuring that the AI analyzes movement patterns without access to personal identity. The identifying features can include facial features, tattoos, birthmarks, scars, distinctive physical markings, and combinations thereof. In certain embodiments, the video recording 116 can be processed to obscure identifying features of the user prior to analysis by the image recognition model 132. The obscuring may be accomplished through techniques such as pixelation (replacing regions with uniform pixel blocks), blurring (applying Gaussian blur to specific regions), masking (overlaying opaque shapes over regions), or feature removal (digitally removing the features entirely). The system 100 can employ automated detection to identify which regions of the video recording 116 contain identifying features. For example, facial recognition algorithms may detect faces and automatically blur or pixelate facial regions. Similarly, the system 100 may detect regions likely to contain tattoos and apply obscuring techniques to those regions.

In certain embodiments, the system 100 may maintain two versions of the video recording 116: an obscured version transmitted to the image recognition model 132 for automated or instructor analysis, and a non-obscured version stored in the database 140 for instructor and/or group administrator review via the instructor interface. This dual-version approach can ensure that the image recognition model 132 analyzes movement patterns without access to identifying information, while instructors retain the ability to view the complete, unobscured video recording 116 for clinical assessment 120 purposes.

Data transmitted to external AI services can be strategically limited to protect proprietary information contained in the database 140. For example, the large language model 130 may be trained to analyze limited information, including binary inputs, such as “1” indicating that a movement 118 shown in a video recording 116 can be complete, and “0” indicating that a movement 118 shown in a video recording 116 can be incomplete. The assessment and education platform 106 can then store responses 146 in the database 140 for future use.

In certain embodiments, the system 100 can anonymize user data for storage in the database 140 or a secondary database 140' for research, educational, and third-party sharing purposes. The anonymization process can include removing all identifying information from the data, including identification information 136 such as name, ID number, and contact information, and demographic information 138 such as age and gender, prior to storage in the secondary database. The anonymized data can include video recordings 116, assessments 120, grades 122, movement improvement metrics, and lesson completion data with all user identifying information removed, ensuring that individual users cannot be identified from the anonymized dataset. The anonymized data stored in the secondary database can be shared with third parties such as research institutions, academic organizations, healthcare systems, and other entities for purposes such as population-level movement quality research, development and improvement of clinical assessment standards, training and refinement of AI models including the image recognition model 132 and large language model 130, and advancement of movement literacy education. The anonymization process can be performed automatically by the system 100 prior to any third-party data sharing, ensuring that user privacy can be protected at all times and that no identifying information can be disclosed to external parties without the explicit consent of the user. The system 100 can implement technical safeguards to verify that anonymization can be complete and accurate before data can be transmitted to the secondary database or shared with third parties, maintaining compliance with applicable data privacy regulations and protecting the confidentiality of all users of the assessment and education platform 106.

In certain embodiments, the system 100 can be accessible via a device type selected from a group consisting of desktop computers, laptop computers, tablet devices, and mobile smartphones. The assessment and education platform 106 can be implemented as a web-based application accessible through standard web browsers, or as a native application that can be downloaded and installed on user devices. The system 100 can be executed through software stored on a computer with a processor 104, a memory 102, and a local or remote server 144 that can facilitate the assessment and education platform 106 via a network. The implementation of the assessment and education platform 106 may be downloaded or installed as an application or plugin associated with an application on the computer. A computer may include any device in communication with a network having a processor 104, a memory 102 having the ability to store software in the form of processor-executable instructions, a camera suitable for capturing video, and a display suitable for displaying video received from the camera or over a network such as the internet or a local area network from another computer. The network can include the internet, local area networks, wide area networks, cellular networks, or any combination thereof. Communication between components of the system 100 can occur over the network using standard networking protocols.

In certain embodiments, ways of configuring and using the system 100 for clinical assessment of a physical movement of a user and progressive improvement of the physical movement can include guidance by one or more instructors having various backgrounds and expertise. The instructor can include one or more of a physical therapist, a physical education teacher, a medical professional, a class instructor, and a coach. The system 100 can be configured to support various types of instructors with different areas of expertise and different use cases. Physical therapists can use the system 100 for remote patient monitoring and rehabilitation program delivery. Physical education teachers can use the system 100 to assess and educate students in school settings. Medical professionals such as sports medicine physicians can use the system 100 to monitor patient movement quality as part of comprehensive care. Class instructors such as yoga or fitness instructors can use the system 100 to provide feedback 126 to students. Coaches can use the system 100 to assess and improve athlete movement patterns.

The physical movement 118 can include one or more various exercises, limb movements, whole body movements, where particular examples include a bodyweight squat, a forward lunge, a hinge, a standing rotation, a modified pushup, a modified plank, and a modified pullup. These movements can be selected to assess fundamental movement patterns that can be important for overall physical function and injury prevention. A bodyweight squat can assess lower body strength, hip strength and flexibility, knee mobility, spinal flexibility and stability, ankle mobility, and balance. A forward lunge can assess single-leg stability, foot position, hip strength and flexibility, spinal flexibility and stability, knee mobility, leg strength, and dynamic balance. A hinge can assess posterior chain strength, hip mobility, and spinal strength and stability. A standing rotation can assess thoracic mobility, core stability, and rotational control. A modified pushup can assess upper body strength, shoulder stability, and core strength. A modified plank can assess core stability, postural alignment, and shoulder stability. A modified pullup can assess upper body pulling strength, postural stability and alignment, and shoulder stability. The system 100 can support assessment 120 of these and other physical movements, and the database 140 can include reference data and training data 141 for a wide variety of movement patterns beyond those specifically enumerated herein.

In certain embodiments, the final grading data 139 can include individual scores for multiple movement characteristics, and the prioritized sequence of lessons 128 can be based on which movement characteristics received lowest scores. This approach can ensure that educational content addresses the user’s most significant deficiencies first, allowing for the largest impact in injury prevention and maximized motor learning.

In certain embodiments, the assessment 120 can evaluate movements on various characteristics including posture, spinal alignment, body angle, foot positioning, knee tracking, hip positioning, shoulder positioning, balance, stability, flexibility, range of motion, joint angles, movement symmetry, and center of mass displacement. Each of these characteristics can be scored individually, allowing for detailed assessment 120 of movement quality.

Posture can refer to the overall alignment of the body during the movement. Spinal alignment can refer to whether the spine maintains appropriate curvature(s). Body angle can refer to the angle of the torso relative to vertical or other reference lines. Foot positioning can refer to placement and orientation of the feet. Knee tracking can refer to the path of the knee during the movement and whether it stays aligned over the foot. Hip positioning can refer to the position and movement of the hips. Shoulder positioning can refer to the position and movement of the shoulders.

Balance can refer to the ability to maintain stability during the movement. Stability can refer to control of body position and resistance to unwanted movement. Flexibility can refer to range of motion at joints. Range of motion can refer to the extent of movement possible at a joint. Joint angles can refer to specific angular measurements at joints during movement. Movement symmetry can refer to whether left and right sides of the body move similarly. Center of mass displacement can refer to movement of the body’s center of mass during the exercise.

The system 100 can provide analytical capabilities that can extend beyond the scope of manual human observation and assessment. The image recognition model 132 can analyze a plurality of video recordings 116 simultaneously, drawing upon analysis of a vast corpus of movement data stored in the database 140 that can encompass millions of physical movements 118. This breadth of analytical experience can enable the image recognition model 132 to identify movement characteristics, patterns, and deficiencies that may not be detectable through manual human observation alone, including subtle biomechanical deviations, compensatory movement patterns, and early indicators of musculoskeletal imbalance that may not yet manifest as pain or functional limitation for the user.

The image recognition model 132 can be configured to analyze both subject physical movements 118 and non-subject physical movements within the video recording 116, detecting movement patterns and characteristics that may occur incidentally during the execution of the prescribed physical movement 118. For example, the image recognition model 132 can detect compensatory movements in body segments not directly targeted by the prescribed movement 118, such as shoulder elevation during a lower body movement or lateral trunk shift during a bilateral exercise. These incidental movement patterns can provide clinically valuable information about the user's overall movement quality and potential areas of concern that may not have been identified by the user or actively sought by the instructor during the review process.

The image recognition model 132 can therefore serve as a comprehensive movement analysis tool that can supplement the instructor's clinical expertise by identifying movement characteristics that the human eye may not detect during routine video review. Issues not reported by the user and not actively sought by the instructor can be identified and flagged by the image recognition model 132 for instructor consideration, enabling a more thorough and comprehensive assessment 120 than may be achievable through manual review alone. This capability can ensure that users receive a complete and accurate evaluation of their movement quality, supporting earlier identification of potential issues and more targeted educational interventions through the education module 112.

In addition to the system embodiments described above, the present technology can include method embodiments for clinical assessment of a physical movement 118 of a user and progressive improvement of the physical movement 118 guided by an instructor and/or group administrator. These methods can leverage the system 100 components and artificial intelligence capabilities, including both the image recognition model 132 and large language model 130, to provide a comprehensive, iterative approach to movement assessment and education.

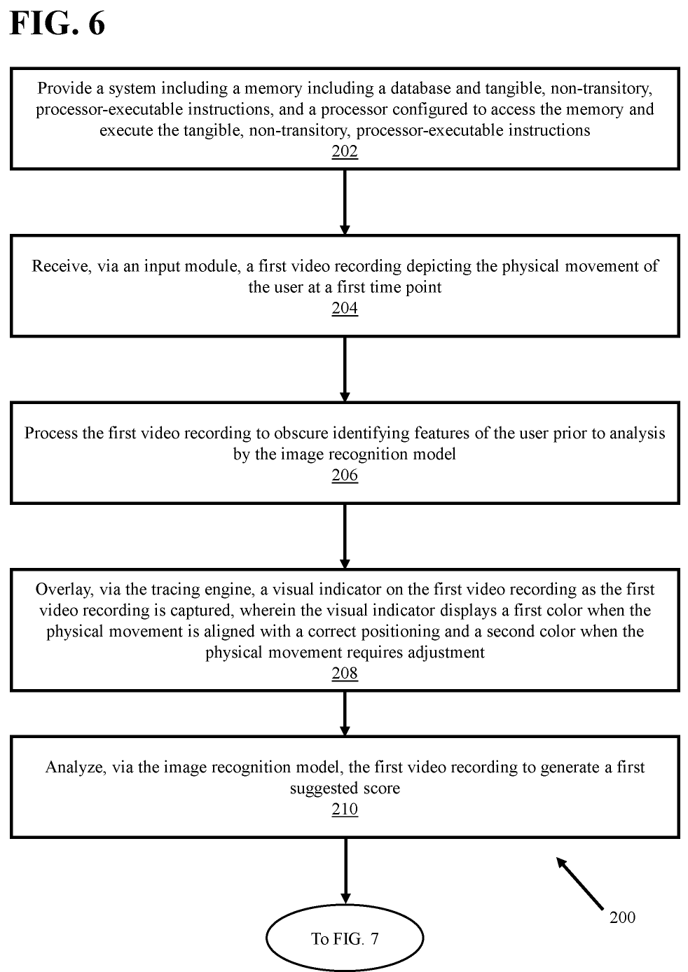

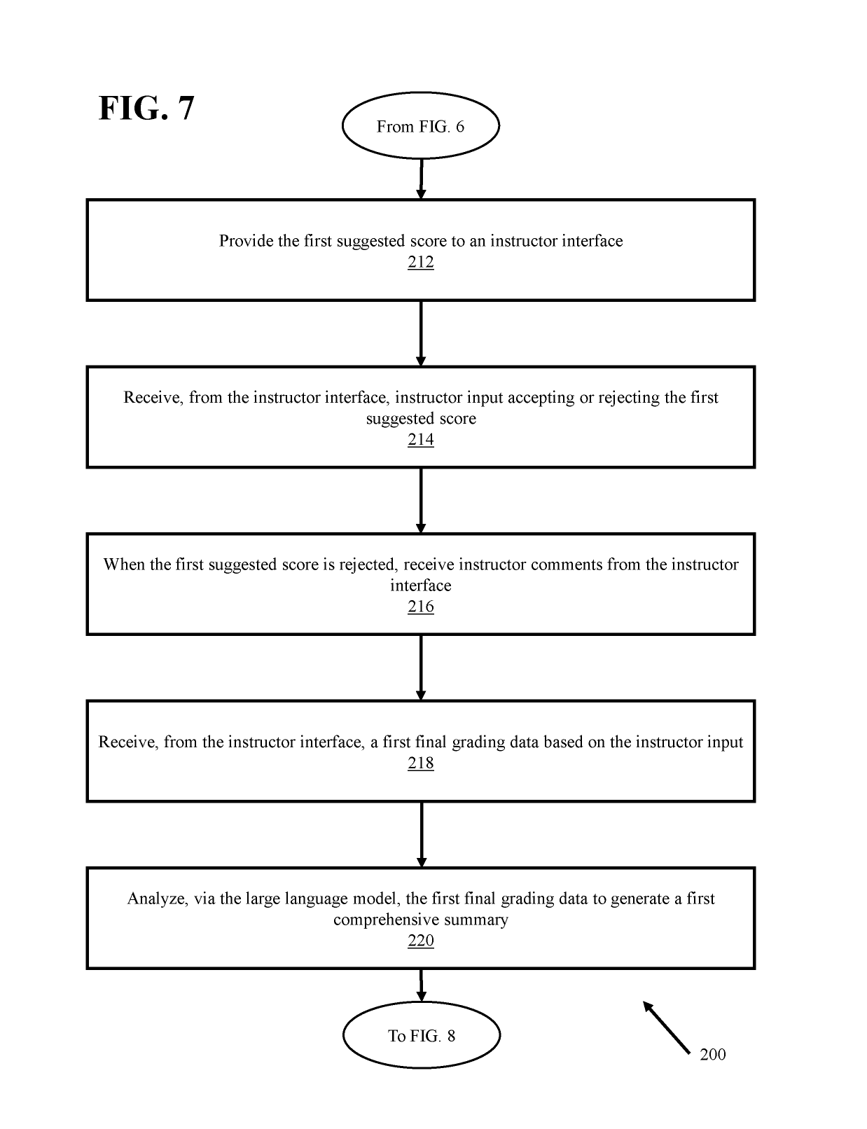

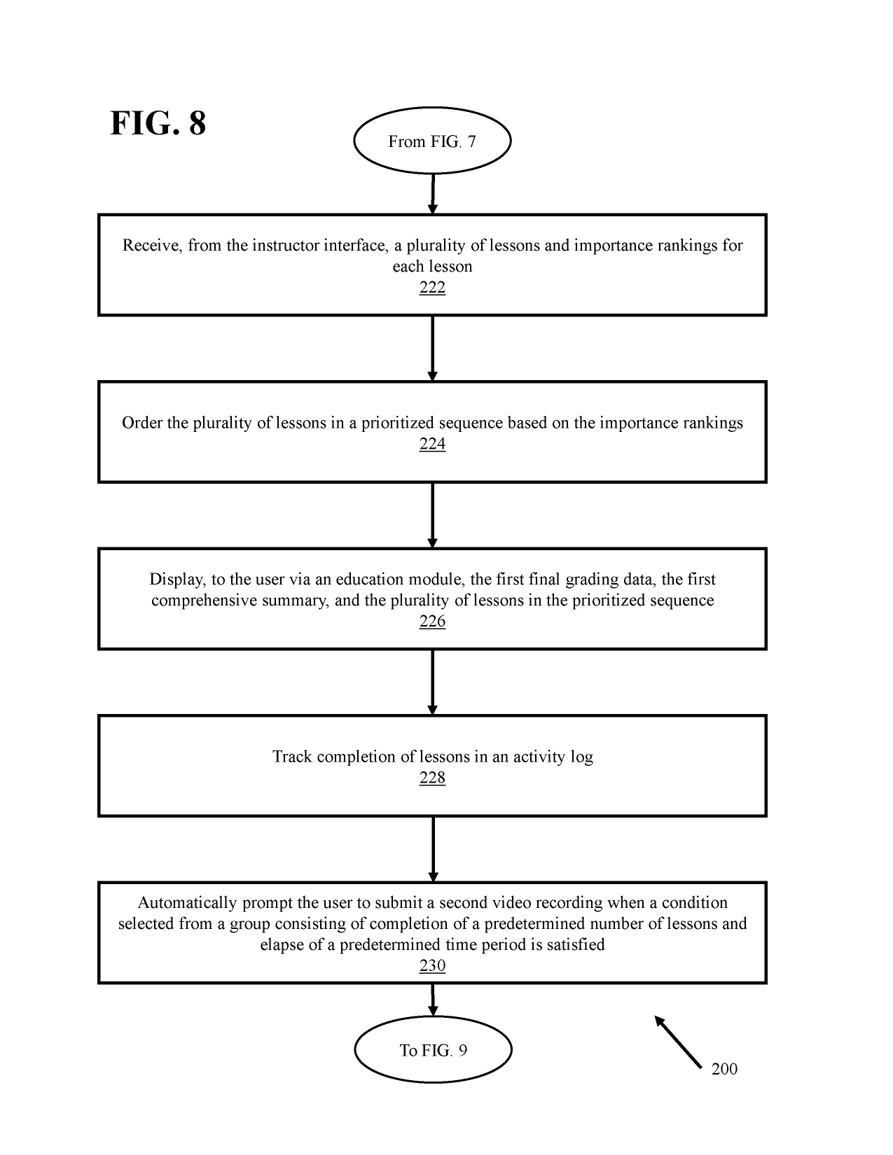

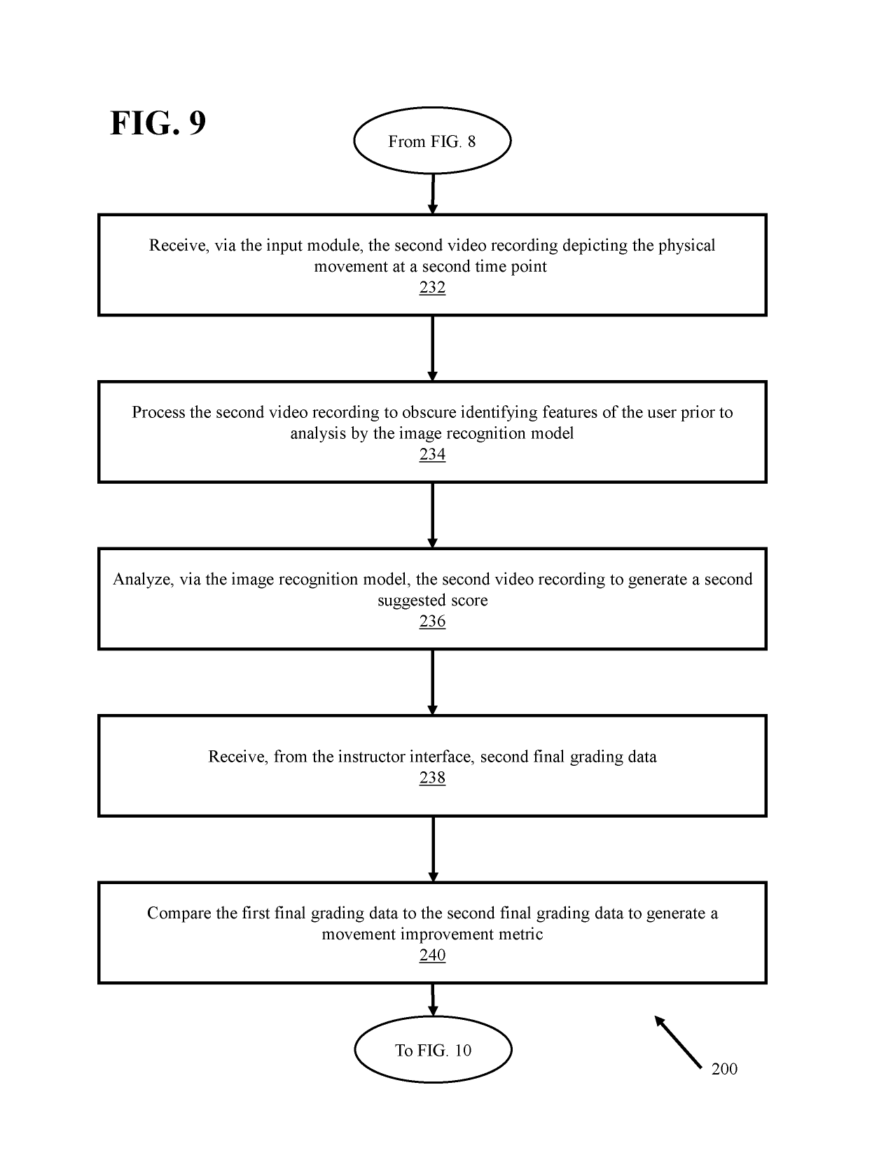

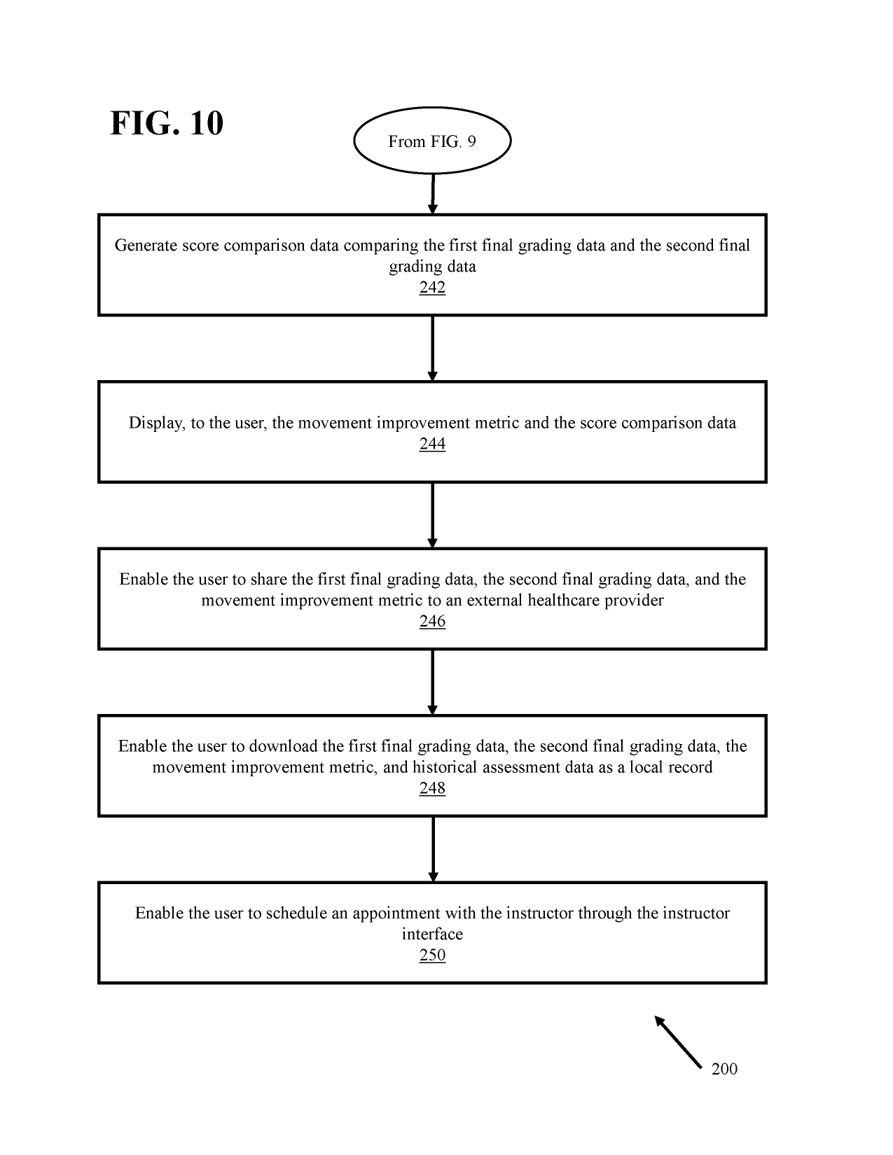

Referring now to FIGS. 6-10, a method 200 for clinical assessment and education of physical activities can be provided. The method 200 can include a series of steps that can be performed using the system 100 as previously described. While the steps may be described in a particular sequence, it should be appreciated that certain steps can be performed simultaneously, in different orders, or iteratively depending on the specific implementation and user interaction patterns.

The method 200 can include providing a system 100 as described herein. The system 100 can include providing a memory 102, a processor 104 coupled with the memory 102, and an assessment and education platform 106. The memory 102 can include a database 140 storing training data 141, assessment data, historical assessment data, and educational content. The memory 102 can also include processor-executable instructions including an image recognition model 132, a large language model 130, a tracing engine 134, and a library 148. The processor 104 can be configured to access the memory 102 and execute the processor-executable instructions. The assessment and education platform 106 can include an input module 108, an assessment module 110, and an education module 112. Both the image recognition model 132 and the large language model 130 can be trained on the database 140 as previously described.

The method 200 can include receiving data 114 from the user via the input module 108 on the assessment and education platform 106. The data 114 can include identification information 136 and demographic information 138 of the user. The identification information 136 may include the user’s name, a unique identifier or ID number assigned by the system 100, and contact information. The demographic information 138 may include age, gender, height, weight, activity level, medical history, current physical condition, and goals or objectives for using the system 100. This data 114 can be entered by the user through a user interface displayed on a computer, which may be a desktop computer, laptop, tablet, or smartphone.

The method 200 can include receiving a video recording 116 from the user via the input module 108. The video recording 116 can depict a physical movement 118 of the user. The physical movement 118 may include a bodyweight squat, forward lunge, hinge, standing rotation, modified pushup, modified plank, modified pullup, or other movement selected for assessment. The video recording 116 can be captured using a camera associated with the computer, such as a built-in webcam, smartphone camera, or external camera. The user can position the camera to capture a full-body view of the movement 118 execution, and can record themselves performing the movement 118 according to instructions provided by the assessment and education platform 106.

The video recording 116 can be uploaded to the input module 108 through the user interface. The upload process may involve selecting a previously recorded video file from the user’s device, or recording video directly through the assessment and education platform 106 interface. Once uploaded, the video recording 116 can be stored in the memory 102 and made available for analysis by the image recognition model 132 and review by the instructor.

The method 200 can include overlaying, via the tracing engine 134, a visual indicator 137 on the video recording 116 in real-time as the video recording 116 can be captured. This step can occur simultaneously with the video recording capture, providing immediate feedback to the user during movement execution. The tracing engine 134 can process the video recording 116 frame-by-frame as frames can be captured, identifying anatomical keypoints in real-time and comparing their positions to reference positions stored in the database 140.

The visual indicator 137 can display a first color when the physical movement 118 can be aligned with a correct positioning and a second color when the physical movement 118 can require adjustment. In certain embodiments, the first color can be green and the second color can be red, providing intuitive visual feedback that the user can immediately understand without needing to interpret complex textual or numerical information. The visual indicators 137 may include dots positioned at anatomical keypoints, lines connecting keypoints to show body segment alignment, arrows indicating direction of correction, and combinations thereof.

This real-time visual feedback can enable the user to make corrections during movement execution rather than only learning about errors after the movement can be complete. For example, if a user can be performing a bodyweight squat and their knees track too far forward, red visual indicators 137 can appear on the live video feed showing the incorrect knee position, allowing the user to adjust their form immediately and attempt the movement again with proper positioning.

The method 200 can include analyzing, via the image recognition model 132, the video recording 116 to generate a suggested score. This analysis can occur after the video recording 116 has been uploaded, and can be performed automatically by the system 100 without requiring manual initiation by the user or instructor. The video recording 116 can be processed before transfer to the image recognition model 132 to protect user privacy, potentially including obscuring identifying features such as facial features, tattoos, birthmarks, scars, and distinctive physical markings through techniques such as pixelation, blurring, masking, or feature removal.

The image recognition model 132 can analyze the video recording 116 frame-by-frame, processing frames at a rate that may be 30 to 60 frames per second or higher. For each frame, the image recognition model 132 can identify anatomical keypoints such as head, spine, shoulders, elbows, wrists, hips, knees, and ankles using pose estimation algorithms. The image recognition model 132 can calculate positions of these anatomical keypoints throughout the movement 118, determine joint angles between body segments using vector mathematics, and track how these angles change throughout the movement execution.

The image recognition model 132 can compare observed movement patterns to reference data stored in the database 140 that defines correct positioning for the specific movement 118 being assessed. The reference data can include acceptable ranges for joint angles, body segment positions, and other biomechanical parameters at various phases of each movement. The image recognition model 132 can identify deviations from these reference ranges and calculate how significant the deviations may be.

Based on this analysis, the image recognition model 132 can generate a suggested score 135 that may include numerical scores for various movement criteria such as posture, spinal alignment, body angle, foot positioning, knee tracking, hip positioning, shoulder positioning, balance, stability, flexibility, range of motion, joint angles, movement symmetry, and center of mass displacement. The suggested score 135 may also include binary completion indicators showing whether the user successfully performed the movement, individual scores for multiple movement criteria assessed separately, or combinations thereof. The suggested score 135 and associated analysis results can be transmitted to the assessment module 110 for incorporation into the assessment 120.

The method 200 can include assessing the data 114, the response from the image recognition model 132, and the video recording 116 via the assessment module 110 to generate an assessment 120. The assessment module 110 can combine the automated analysis from the image recognition model 132 with the user’s demographic and identification data 114 to create a comprehensive assessment 120 package for instructor and/or group administrator review. The assessment 120 can present the suggested scores alongside the video recording 116, with visual indicators 137 highlighting the specific movement characteristics that led to each suggested score.

The method 200 can include providing the assessment 120 to the instructor. The assessment 120 can be transmitted from the assessment module 110 to an instructor interface, which can be accessed by the instructor through their own computer connected to the system 100 via a network. The instructor interface can display the assessment 120 in a structured format that presents the video recording 116, the suggested score 135 from the image recognition model 132, the user’s data 114, and any relevant historical assessment data if the user has been previously assessed. The instructor interface 123 can display the suggested score 135 from the image recognition model 132 with visual indicators 137 highlighting areas of concern in the video recording 116. The instructor can review the assessment 120 by watching the video recording 116, examining the suggested scores for each movement criterion, and applying their clinical expertise to evaluate the user’s movement quality. The instructor interface can provide tools for the instructor to annotate the video recording 116, highlight specific issues, and add notes about observed movement patterns or deficiencies.

The method 200 can include receiving instructor input accepting or rejecting the suggested score. The instructor may accept the suggested score 135 without modification if the AI analysis can be accurate, reject the suggested score 135 if the AI analysis can be incorrect, or modify the suggested score 135 to adjust specific criteria. This human-in-the-loop validation can ensure clinical accuracy while leveraging the efficiency of automated analysis.

The method 200 can include receiving instructor comments when the suggested score 135 can be rejected. When the instructor rejects or modifies the suggested score, the instructor interface can prompt the instructor to provide textual comments explaining the rejection or modification. These instructor comments can provide valuable context for understanding why the AI suggestion was incorrect and can be used to continuously improve the image recognition model 132 over time through retraining with additional annotated data.

The method 200 can include receiving final grading data 139 from the instructor interface based on the instructor input. The final grading data 139 can represent the instructor’s validated assessment 120 of the user’s movement quality, incorporating the instructor’s clinical expertise and judgment. The final grading data 139 can include numerical scores for various movement criteria, overall movement quality ratings, identification of specific movement deficiencies, and qualitative observations about the user’s performance.

The method 200 can include analyzing, via the large language model 130, the final grading data 139 to generate a comprehensive summary. Once the instructor provides final grading data 139, the assessment module 110 can transmit this data to the large language model 130 for analysis and summary generation. The data transmitted to the large language model 130 may be strategically limited to protect proprietary information and user privacy, potentially including only the numerical scores, identified deficiencies, and instructor comments without the associated video content or detailed user identifying information.

The large language model 130 can analyze the final grading data 139 in the context of information stored in the database 140, including clinical knowledge about movement mechanics and common movement deficiencies, previously graded assessments with corresponding expert-written summaries that served as training data 141, information about appropriate corrective interventions for specific movement problems, and patterns observed across multiple users with similar deficiencies. The large language model 130 can identify which aspects of the user’s performance can be strengths, which aspects can be deficiencies requiring attention, how the identified deficiencies may impact the user’s function or injury risk, and what educational interventions may be most appropriate.

The large language model 130 can generate a summary that explains the assessment 120 results in clear, understandable language appropriate for the user’s level of knowledge. The summary can describe what the user performed correctly during the movement 118 execution, identify specific areas needing improvement with detailed explanations, explain why certain movement patterns may be problematic in terms of injury risk or functional limitations, provide context for the assigned scores and how they relate to normal movement patterns, relate the findings to the user’s stated goals or medical history from their demographic information 138, and set expectations for the lessons 128 that will be assigned and what improvements can be anticipated.

The summary generated by the large language model 130 can incorporate the instructor comments and can align the summary with the instructor input. If the instructor rejected the suggested score 135 from the image recognition model 132 and provided comments explaining their reasoning, the large language model 130 can integrate this information into the summary, ensuring that the final feedback to the user reflects the instructor’s clinical judgment. The summary can be transmitted back to the assessment module 110 for inclusion in the content displayed to the user.

The method 200 can include receiving a plurality of lessons 128 and importance rankings for each lesson from the instructor interface. The instructor can select one or more lessons 128 from the library 148 based on the movement deficiencies identified in the assessment 120 and reflected in the final grading data 139. The library 148 can contain a plurality of library items 150 organized into categories 152, with each library item 150 addressing specific movement deficiencies or educational topics.

The instructor can search or browse the library 148 to identify appropriate lessons 128 for the user. The instructor interface can provide automated suggestions based on the final grading data 139 and identified deficiencies, recommending library items 150 that have been linked to specific movement deficiency patterns. The instructor can accept these automated suggestions, modify the selection, or choose entirely different lessons 128 based on their clinical judgment and knowledge of the user’s or group administrator’s specific needs and goals.

For each selected lesson 128, the instructor can assign an importance ranking based on factors such as severity of the movement deficiency, risk of injury associated with the deficiency, foundational nature of the movement skill being taught, prerequisite relationships between lessons, and user-specific goals or circumstances. These importance rankings can guide the sequence in which the user should complete the lessons 128. These will be tallied for a group and organized for the group administrator in the order of importance.

The method 200 can include ordering the plurality of lessons 128 in a prioritized sequence based on the importance rankings. The assessment module 110 or education module 112 can automatically sort the selected lessons 128 according to the importance rankings provided by the instructor, creating a structured learning pathway for the user and group administrator.

The method 200 can include displaying the final grading data 139, the summary, and the plurality of lessons 128 in the prioritized sequence to the user via the education module 112. Once the instructor has completed their review, the large language model 130 has generated the summary, and the instructor has selected appropriate lessons 128, the education module 112 can compile this information and present it to the user and/or group administrator through the user interface on the user’s and/or group administrator’s computer.

The display can include the final grading data 139 showing scores for each movement criterion assessed, the summary generated by the large language model 130 (which incorporates the instructor’s comments and clinical judgment), the plurality of lessons 128 ordered in a prioritized sequence based on the importance rankings assigned by the instructor, and any additional feedback 126 or guidance provided by the instructor. The user and/or group administrator can access this information by logging into the assessment and education platform 106.

The method 200 can include tracking completion of lessons 128 in an activity log. The education module 112 can maintain an activity log that records detailed information about user interaction with educational content. For each lesson 128, the activity log can store a timestamp indicating when the lesson 128 was assigned, a timestamp indicating when the user began accessing the lesson 128, a duration indicating total time spent on the lesson 128, a timestamp indicating when the lesson 128 was completed, and a completion status indicator showing whether the lesson 128 can be not started, in progress, or completed.

The method 200 can include automatically prompting the user to submit a subsequent video recording 116 when a condition selected from a group consisting of completion of a predetermined number of lessons 128 and elapse of a predetermined time period can be satisfied. The education module 112 can query the activity log to determine whether the predetermined conditions have been met. The predetermined conditions may include completion of all assigned lessons 128, completion of a specific number or percentage of assigned lessons 128 (such as 80% or 5 out of 6 lessons), elapse of a predetermined time period (such as two weeks or one month) since the previous assessment, or a combination of both lesson completion and time elapsed conditions.

When the predetermined conditions can be satisfied, the education module 112 can automatically generate and display a prompt to the user requesting submission of the subsequent video recording 116. The prompt can include contextual information such as which lessons were completed, how much time has elapsed since the previous assessment, expected improvements based on the completed lessons, and encouragement to record the new video to track progress. The prompt may be displayed when the user logs into the assessment and education platform 106, or may be sent to the user via email, text message, or push notification to their device.

The method 200 can include receiving, via the input module 108, the subsequent video recording 116 depicting the physical movement 118 at a second time point. When the user responds to the automated prompt by recording and uploading a new video, the system 100 can receive this subsequent video recording 116 through the same process as the initial video recording 116. The subsequent video recording 116 can depict the same physical movement 118 that was assessed in the initial assessment 120, allowing for direct comparison of movement quality before and after the educational intervention.

The method 200 can include analyzing, via the image recognition model 132, the subsequent video recording 116 to generate a second suggested score. The analysis process can follow the same procedure as the initial analysis, with the image recognition model 132 processing the video frame-by-frame, identifying anatomical keypoints, calculating joint angles, and comparing to reference data.

The method 200 can include receiving second final grading data 139 from the instructor interface. The instructor can review the second assessment in the same manner as the first assessment, examining the suggested score 135 from the image recognition model 132 and applying their clinical expertise to validate or modify the scores. The large language model 130 can analyze the second final grading data 139 to generate a second summary following the same process as the initial summary.

The method 200 can include comparing the first final grading data 139 to the second final grading data 139 to generate a movement improvement metric 143. The assessment module 110 can retrieve the first final grading data 139 from the historical assessment data stored in the database 140 and compare it to the second final grading data 139. The comparison can calculate changes in scores for each movement criterion, determine whether each criterion improved, declined, or remained unchanged, identify persistent deficiencies that did not improve despite completion of assigned lessons 128, and calculate overall improvement metrics.

The movement improvement metrics 143 may include change in overall movement quality score, change in specific movement criteria scores (such as “hip flexibility improved by 2 points” or “knee alignment improved from 5/10 to 8/10”), percentage improvement calculation (such as “15% improvement in overall score”), rate of improvement over time (such as “0.5 points per week improvement rate”), and comparison to population benchmarks (such as “improvement rate can be 20% faster than average for your age group”).

The method 200 can include generating score comparison data comparing the first final grading data 139 and the second final grading data 139. The score comparison data can include a side-by-side numerical comparison of scores from the two assessments, visual highlighting of improved movement criteria (such as displaying improved scores in green text), visual highlighting of persistent movement deficiencies (such as displaying unchanged or declined scores in red or yellow text), and a net improvement calculation showing the overall change in movement quality.

The method 200 can include displaying, to the user and/or group administrator, the movement improvement metric 143 and the score comparison data. The education module 112 can present this information through visual representations such as line graphs showing score trends over time, bar charts comparing scores at the two time points, progress indicators showing advancement toward clinical benchmarks, or textual explanations of what the metrics mean. This display can provide motivational feedback to the user and help them understand the effectiveness of the educational intervention and their own efforts.

Based on the movement improvement metrics 143, the education module 112 can automatically adjust the educational content for subsequent learning. When improvement metrics indicate progress in a particular area, the system 100 can advance the user to more challenging lessons 128 that build on the improved skills. When improvement metrics indicate persistent deficiencies despite completion of assigned lessons 128, the system 100 can provide alternative or supplemental lessons 128 using different teaching approaches or exercises. The instructor can review the movement improvement metrics 143 and make manual adjustments to the automatically suggested content changes, ensuring that the updated educational plan can be clinically appropriate for the user’s needs.

The method 200 can accommodate group-based assessment and education. The method 200 can include creating a group 156 that includes a plurality of user contacts. Creating the group 156 can include receiving a group name from a group administrator (who may be the instructor or a designated administrative user), assigning existing user contacts to the group 156 or creating new user contacts, associating video recordings 116, assessments 120, and lessons 128 with the group 156, and maintaining group membership data in the memory 102.

The method 200 can include receiving data 114 from each user in the group 156, receiving video recordings 116 from each user in the group 156, and analyzing each video recording 116 via the image recognition model 132 to generate suggested scores for each user. The image recognition model 132 can process multiple video recordings in parallel or sequentially, generating suggested scores for each user following the same analysis procedure described above.

The method 200 can include providing the assessments 120 to the instructor via an instructor interface configured for group management. The instructor interface can display assessments 120 for all users in the group 156, allowing the instructor to review multiple users efficiently. The instructor can receive tools for comparing assessments 120 across users, identifying common patterns or deficiencies affecting multiple users, and managing the grading process for the entire group 156. A group administrator can also access the group 156 assessments 120 via a group administrator interface. The group administrator interface can display assessments 120 for all users in the group 156, allowing the group administrator to review and compare assessments 120 across users and identify common patterns or deficiencies affecting multiple users in the group 156. Unlike the instructor, the group administrator may not manage the grading process but can utilize the comparative assessment data to inform group programming and instructional decisions.

The method 200 can include receiving final grading data 139 for each user from the instructor interface. The instructor can grade each user individually, or can apply consistent grading criteria across the group 156 to ensure fairness and comparability. The large language model 130 can generate summaries for each user following the same process described above.

The method 200 can include calculating an average score for the group 156 based on individual final grading data 139 from the plurality of users. The assessment module 110 can aggregate the final grading data 139 from all users in the group 156 and calculate mean scores for each movement criterion. The method 200 can include identifying common movement deficiencies present across multiple users in the group 156. The assessment module 110 can analyze the final grading data 139 to determine which deficiencies can be most prevalent in the group 156.

The method 200 can include tallying lesson assignments across the plurality of users to determine which lessons 128 can be assigned most frequently. The assessment module 110 can count how many users in the group 156 have been assigned each lesson 128 from the library 148. The method 200 can include ordering lessons 128 for the group 156 in a group prioritized sequence based on the tallying, wherein lessons 128 needed by a majority of users in the group 156 can be prioritized higher.

The method 200 can include displaying the average score, individual scores for each user, and the group prioritized sequence to a group administrator interface. The group administrator interface can present group statistics including the average score for the group 156, distribution of scores across different movement criteria, identification of the most common deficiencies affecting the group 156, and a prioritized list of lessons 128 that would benefit the majority of users in the group 156. This group-level view can help the instructor design effective group programming while also maintaining the ability to provide individualized instruction where needed.

The system 100 and the method 200 can include in-person training and physical resources to facilitate the efficient and effective completion of prescribed exercise routines and associated movements 118. The in-person training can include qualified professionals such as coaches, physical therapists, and rehabilitation specialists who can observe users directly and provide real-time feedback during movement execution. The in-person training can provide certification opportunities for instructors and group administrators seeking to deliver the method in professional settings.

The in-person training can result in personalized guidance to enhance overall physical longevity and instruct physical literacy, movement literacy, and body awareness by incorporating techniques that promote functional movement, joint mobility, and stability. The method can include supplemental educational materials such as participant manuals and education manuals to reinforce learning content for instructors, group administrators, and users. The participant manual can include pictorial content and blank spaces for note-taking, allowing users to engage actively with the material and record personalized observations. The education manual can include fully written, scientifically based content explaining why movement literacy can be beneficial and how to achieve it, and can discuss how to apply information gathered in the assessment and education platform 106 to supplement specific applications of the instructor or group administrator.

The method 200 can include physical visual aids such as a spine model to demonstrate the function of the spine, pelvis, and shoulder girdle to further assist in the learning process of the user and provide tools for the group administrator and instructor to use during in-person training sessions. The spine model can provide a tangible, three-dimensional representation of anatomical structures that can be referenced during instruction to enhance understanding of movement mechanics and the clinical basis for prescribed exercises and movements 118.

The system 100 and the method 200 can integrate clinical expertise with technology and physical resources to help users maintain long-term physical health and improve overall well-being. The various tools, including the assessment and education platform 106, in-person training, supplemental educational materials, and physical visual aids, can ensure that all learning styles can be addressed and supported, including visual learning through the assessment and education platform 106 displays and visual aids, auditory learning through in-person instruction and video content, and kinesthetic learning through physical practice of prescribed movements 118 and exercises.

The method 200 can include in-person training sessions configured to provide certification in the method 200 and comprehensive understanding of how the system 100 and its components function. The in-person training sessions can establish a collaborative relationship between instructors and group administrators, enabling effective delivery of the method 200 across a variety of professional settings. The instructors can include medical providers such as physical therapists, physicians, and athletic trainers who can provide clinical oversight, validate assessments 120, and ensure that prescribed movements 118 and lessons 128 can be clinically appropriate for each user. The group administrators can include physical education teachers, health teachers, coaches, and trainers who can apply the method 200 in educational and athletic settings, leveraging the assessment and education platform 106 to deliver clinically informed movement education to groups and individuals. The collaborative relationship between instructors and group administrators can bridge the gap between clinical expertise and practical application, ensuring that users can receive both medically grounded guidance and accessible, day-to-day movement instruction.

The method 200 can also include physical resources to supplement the digital content available through the assessment and education platform 106. The physical resources can include a spine model with ligaments configured to demonstrate the function of the spine, pelvis, and shoulder girdle, providing a tangible, three-dimensional anatomical reference that can enhance understanding of movement mechanics during in-person training sessions. The physical resources can also include books configured to function as training manuals for instructors and group administrators and as participant manuals for users. The training manuals can include fully written, scientifically based content explaining the clinical basis for movement literacy and how to apply information gathered through the assessment and education platform 106 in specific professional settings. The participant manuals can include pictorial content and blank spaces for note-taking, allowing users to engage actively with the material and record personalized observations. The books can be configured to align with and supplement the library 148, library items 150, categories 152, and lessons 128 contained in the assessment and education platform 106, providing users, group administrators, and instructors with a comprehensive set of physical and digital resources that can address all learning styles, including visual, auditory, and kinesthetic learning.

### EXAMPLES

Example embodiments of the present technology are provided with reference to the several figures enclosed herewith.

Example 1: Personalized Physical Assessment for an Office Worker

An office worker spends long hours seated at their desk, leading to chronic lower back pain and poor posture. Using the input module 108 on the assessment and education platform 106, they upload a video recording 116 where they can be shown executing a forward lunge. The system 100 utilizes the image recognition model 132 to complete an initial assessment 120 that evaluates their posture, flexibility, and range of motion. The system 100 analyzes their movement patterns via the assessment module 110 and prescribes a personalized routine focusing on spine support, stabilization, and stretching exercises to counteract prolonged sitting. The personal routine can come in the form of a lesson 128, with various applicable library items 150, and can be accessed by the office worker via the education module 112.

The worker may also be video recorded 116 performing a set of movements 118, for example, modified pushups, modified pullups, and modified planks. The image recognition model 132 analyzes their movements 118 and generates a response 146. The video recording 116 and the response 146 from the image recognition model 132 can be checked by an instructor for customized feedback 126 and lessons 128, ensuring gradual improvement in posture and pain reduction.

Example 2: Balance and Stability Program for Seniors

A group 156 of individuals living in a retirement community may be experiencing issues with balance, making them prone to falls. A wellness coach uploads video recordings 116 of each individual executing various movements 118 to the input module 108 on the assessment and education platform 106, which can be downloaded onto the laptop that the coach uses to record the individuals.

The assessment module 110 on the assessment and education platform 106 allows the instructor to grade 122 the video recording 116 of each individual and recommends targeted exercises such as a bodyweight squat, a forward lunge, a hinge, a standing rotation, a modified pushup, a modified plank, a modified pullup, and other movements 118 for stabilizing muscles, all with accompanying lessons 128 that the wellness coach may demonstrate to the group 156 or provide to each individual separately via the education module 112. For example, the assessment module 110 may include a tracing engine 134 that superimposes dots, lines, and arrows over each individual’s torso and limbs in the video recording 116 to give a live assessment 120 showing when each individual’s movement 118 can be correct.

Example 3: Clinical Rehabilitation for a Post-Surgery Patient

A patient recently undergoes knee surgery and needs a structured rehabilitation program. The input module 108 on the assessment and education platform 106 allows them to upload a video recording 116 showing them performing various movements 118 and connects them with a virtual physical therapist that may assess the movements 118 via the assessment module 110 with the help of the image recognition model 132 and recommend customized lessons 128 and assign progressive exercises and library items 150 for the patient based on their range of motion and pain levels.

The patient logs their progress daily on the education module 112, and the system 100 provides adjustments and feedback 126 as they complete each lesson 128, ensuring a gradual and safe recovery. The patient may also be video recorded performing their rehabilitation exercises, allowing the tracing engine 134 to analyze their range of motion and detect deviations from the prescribed movements 118, and offer personalized guidance to optimize their recovery process.

Example 4: Longitudinal Progress Tracking for a Rehabilitation Patient

A patient recovering from knee surgery uploads an initial video recording 116 performing a bodyweight squat. The assessment module 110 analyzes the video recording 116 and identifies limited knee flexion (achieving only 60 degrees) and forward knee tracking indicating weak hip stabilizers. An instructor reviews the assessment 120, provides a grade 122 confirming these deficiencies, and assigns lessons 128 focused on knee range-of-motion exercises and hip strengthening.

Over the next four weeks, the patient completes the assigned lessons 128, which the education module 112 tracks. At the end of week four, the patient uploads a second video recording 116 of the same bodyweight squat movement 118. The assessment module 110 generates a new assessment 120 and compares it to the initial assessment 120. The comparison shows improved knee flexion (now achieving 75 degrees, a 15-degree improvement) but persistent forward knee tracking (minimal improvement in hip stability).

The system 100 displays these improvement metrics to the patient via the education module 112, showing a graph of knee flexion improvement over time. Based on the improvement metrics, the education module 112 automatically adjusts the educational content: it advances the patient to more challenging knee mobility exercises (since knee flexion can be improving) while providing additional hip stability lessons 128 (since hip stabilization remains deficient).

Eight weeks later, the patient uploads a third video recording 116. The assessment 120 now shows knee flexion of 135 degrees (clinical normal) and significantly improved knee tracking (proper hip stabilization). The system 100 calculates overall improvement metrics showing 60-degree improvement in knee flexion and 80% reduction in forward knee tracking over the 12-week period. The education module 112 displays a progress report showing the patient’s improvement trajectory, correlating specific improvements with completed lessons 128, and indicating that the patient has achieved clinical recovery benchmarks. This longitudinal tracking and automated content adjustment enabled the patient to achieve measurable improvement through a data-driven rehabilitation process.

Example 5: Iterative Improvement for Athletic Training

A high school athlete uploads a video recording 116 performing a bodyweight squat. The system 100 assesses the movement and identifies limited hip flexibility and forward knee tracking. The instructor grades 122 the assessment 120, confirms the deficiencies, and assigns lessons 128 from the library 148 focusing on hip mobility exercises and proper knee alignment cues.

Two weeks later, after completing the assigned lessons 128 and practicing the corrective exercises, the athlete uploads a new video recording 116 of the same bodyweight squat movement 118. The system 100 generates a new assessment 120 and compares it to the initial assessment 120. The comparison shows improved hip flexion angle (increased from 85 degrees to 105 degrees) and improved knee tracking (reduced medial collapse from 15 degrees to 5 degrees). The system 100 displays these improvement metrics to both the athlete and instructor.

Based on the improved hip flexibility but persistent knee tracking issues, the system 100 automatically prioritizes additional educational content focused on knee stability while advancing the athlete to more challenging hip mobility exercises. This iterative process continues over multiple assessment 120 cycles, with the system 100 tracking long-term trends and adjusting educational content to address persistent deficiencies while advancing areas of demonstrated improvement.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. A system for clinical assessment of a physical movement of a user and progressive improvement of the physical movement guided by an instructor, comprising:
a memory including a database and tangible, non-transitory, processor-executable instructions, the database including training data, assessment data, historical assessment data, and educational content, the tangible, non-transitory, processor-executable instructions including an image recognition model, a large language model, a tracing engine, an assessment and education platform, and a library; and
a processor configured to access the memory and execute the tangible, non-transitory, processor-executable instructions;
wherein:
the image recognition model is trained on the database and configured to: receive a video recording depicting the physical movement of the user, analyze the video recording to identify a movement characteristic, and generate a suggested score based on the identified movement characteristic;
the large language model is trained on the database and configured to: receive grading data, analyze the grading data by referring to the database, and generate a summary based on the analyzed grading data;
the tracing engine is configured to: overlay a visual indicator on the video recording in real-time as the video recording is captured, track performance of the user during the physical movement and ascertain a positioning thereof, display a first color indicator when the positioning includes a correct positioning, and display a second color indicator when the positioning requires adjustment;
the assessment and education platform includes:
an input module configured to receive user identification data, a video recording depicting the physical movement captured at different time points, and historical assessment data;
an assessment module configured to: provide the suggested score from the image recognition model to an instructor interface, receive instructor input accepting or rejecting the suggested score, receive instructor comments when the suggested score is rejected, receive final grading data from the instructor interface based on the instructor input, provide the final grading data to the large language model, receive the summary from the large language model, generate an assessment, compare assessments across the different time points to generate a movement improvement metric, generate score comparison data enabling comparison of final grading data across the different time points, store assessments as the historical assessment data, and enable sharing of the final grading data and the movement improvement metric to an external healthcare provider;
an education module configured to: receive a plurality of lessons selected using the instructor interface, receive importance rankings for the plurality of lessons from the instructor interface, order the plurality of lessons in a prioritized sequence based on the importance rankings, display the final grading data, the summary, and the plurality of lessons in the prioritized sequence to the user, maintain an activity log tracking a completion status of each lesson, automatically prompt the user to submit a subsequent video recording when a condition selected from a group consisting of completion of a predetermined number of lessons and elapse of a predetermined time period is satisfied, display the score comparison data and the movement improvement metric to the user, enable the user to download the final grading data, the movement improvement metrics, and the historical assessment data as a local record, and enable the user to schedule an appointment with the instructor through the instructor interface; and
the library contains a plurality of library items organized into categories, the plurality of lessons comprise library items selected using the instructor interface from the library based on movement deficiencies identified in the final grading data.

2. The system of claim 1, wherein the instructor interface is configured to: display the suggested score from the image recognition model with a visual indicator highlighting an area of concern in the video recording; receive input from the instructor to accept the suggested score without modification, reject the suggested score, or modify the suggested score; and when the suggested score is rejected or modified, receive textual comments from the instructor explaining the rejection or modification.

3. The system of claim 2, wherein the summary generated by the large language model incorporates the instructor comments and aligns the summary with the instructor input.

4. The system of claim 1, wherein the video recording is processed to obscure identifying features of the user prior to analysis by the image recognition model to protect user privacy, the identifying features comprising a member selected from a group consisting of a facial feature, a tattoo, a birthmark, a scar, a distinctive physical marking, and combinations thereof.

5. The system of claim 1, wherein the database comprises annotated training data including previously graded video recordings with corresponding movement deficiency labels, and both the image recognition model and the large language model are trained on the annotated training data.

6. The system of claim 1, wherein the first color indicator is green and the second color indicator is red.

7. The system of claim 1, wherein the tracing engine is guided by analysis from the image recognition model to determine whether the physical movement is aligned with correct positioning.

8. The system of claim 1, wherein the tracing engine is configured to track performance of the user during the physical movement by: identifying an anatomical keypoint in each frame of the video recording; calculating a position of the anatomical keypoint relative to a reference position stored in the database; and determining alignment based on whether the calculated position is within a predetermined tolerance of the reference position.

9. The system of claim 1, wherein the education module is configured to: determine that the predetermined number of lessons have been completed by querying the activity log, and automatically generate and display a prompt to the user requesting submission of the subsequent video recording.

10. The system of claim 1, wherein the score comparison data comprises: a side-by-side numerical comparison of scores from assessments at different time points, visual highlighting of improved movement criteria, visual highlighting of persistent movement deficiencies, and a net improvement calculation.

11. The system of claim 1, wherein the assessment module is configured to generate a visual representation of the movement improvement metric comprising a member selected from a group consisting of a line graph showing a score trend over time, a bar chart comparing scores at different time points, and progress indicators showing advancement toward a clinical benchmark.

12. The system of claim 1, wherein enabling sharing of the final grading data and the movement improvement metric to an external healthcare provider comprises: generating a shareable report containing the final grading data, the movement improvement metric, and the historical assessment data, encrypting the shareable report, and transmitting the encrypted shareable report to a recipient designated by the user.

13. The system of claim 1, wherein enabling the user to schedule an appointment with an instructor comprises: displaying available appointment times from a calendar associated with the instructor; receiving selection of an appointment time from the user; transmitting appointment confirmation to the user and the instructor; and providing secure video conferencing access at the scheduled appointment time.

14. The system of claim 1, wherein the education module is configured to: display the plurality of lessons in the prioritized sequence with numerical rankings; indicate which lessons are highest priority; and recommend an order of completion based on the prioritized sequence.

15. The system of claim 1, wherein the assessment module is further configured to: create a group including a plurality of user contacts; receive video recordings from each user in the group via multiple video recordings in parallel or sequentially; generate suggested scores for each user via the image recognition model; provide the suggested scores to the instructor interface; receive final grading data for each user from the instructor interface; calculate an average score for the group based on individual final grading data from the plurality of users; identify common movement deficiencies present across multiple users in the group; tally lesson assignments across the plurality of users to determine which lessons are assigned most frequently; and order lessons for the group in a group prioritized sequence based on the tallying, wherein lessons needed by a majority of users in the group are prioritized higher.

16. The system of claim 15, wherein the assessment module is configured to display the average score of the group, an individual score for each user, and the group prioritized sequence to a group administrator interface; and enable one of the instructor and a group administrator to assign lessons to the group, to subgroups, and to individual users.

17. The system of claim 16, wherein the assessment module is configured to: identify a persistent movement deficiency by comparing assessments across multiple time points; determine that a movement deficiency is persistent when scores for a particular movement criterion fail to improve beyond a threshold amount across at least two assessments; and flag the persistent movement deficiency for instructor review.

18. The system of claim 1, wherein the education module is configured to: correlate changes in the movement improvement metric with completed lessons; identify which lessons are most effective for specific movement deficiencies based on the correlation; and prioritize effective lessons for future users with similar movement deficiencies.

19. A method for clinical assessment of a physical movement of a user and progressive improvement of the physical movement guided by an instructor, comprising:
providing the system of claim 1;
receiving, via an input module, a first video recording depicting the physical movement of the user at a first time point;
processing the first video recording to obscure identifying features of the user prior to analysis by the image recognition model;
overlaying, via the tracing engine, a visual indicator on the first video recording as the first video recording is captured, wherein the visual indicator displays a first color when the physical movement is aligned with a correct positioning and a second color when the physical movement requires adjustment;
analyzing, via the image recognition model, the first video recording to generate a first suggested score;
providing the first suggested score to an instructor interface;
receiving, from the instructor interface, instructor input accepting or rejecting the first suggested score;
when the first suggested score is rejected, receiving instructor comments from the instructor interface;
receiving, from the instructor interface, a first final grading data based on the instructor input;
analyzing, via the large language model, the first final grading data to generate a first summary;
receiving, from the instructor interface, a plurality of lessons and importance rankings for each lesson;
ordering the plurality of lessons in a prioritized sequence based on the importance rankings;
displaying, to the user via an education module, the first final grading data, the first summary, and the plurality of lessons in the prioritized sequence;
tracking completion of lessons in an activity log;
automatically prompting the user to submit a second video recording when a condition selected from a group consisting of completion of a predetermined number of lessons and elapse of a predetermined time period is satisfied;
receiving, via the input module, the second video recording depicting the physical movement at a second time point;
analyzing, via the image recognition model, the second video recording to generate a second suggested score;
receiving, from the instructor interface, second final grading data;
comparing the first final grading data to the second final grading data to generate a movement improvement metric;
generating score comparison data comparing the first final grading data and the second final grading data;
displaying, to the user, the movement improvement metric and the score comparison data;
enabling the user to share the first final grading data, the second final grading data, and the movement improvement metric to an external healthcare provider;
enabling the user to download the first final grading data, the second final grading data, the movement improvement metric, and historical assessment data as a local record; and
enabling the user to schedule an appointment with the instructor through the instructor interface.

20. A non-transitory computer-readable medium storing processor-executable instructions that, when executed by a processor, cause the processor to perform a method for clinical assessment of a physical movement of a user and progressive improvement of the physical movement guided by an instructor, the method including:
receiving a first video recording depicting the physical movement of the user at a first time point;
processing the first video recording to obscure identifying features of the user prior to analysis by a image recognition model;
overlaying a visual indicator on the first video recording in real-time as the first video recording is captured, wherein the visual indicator displays a first color when the physical movement is aligned with a correct positioning and a second color when the physical movement requires adjustment;
analyzing, via an image recognition model trained on a database, the first video recording to generate a first suggested score;
providing the first suggested score to an instructor interface;
receiving instructor input accepting or rejecting the first suggested score;
when the first suggested score is rejected, receiving instructor comments from the instructor interface;
receiving first final grading data from the instructor interface based on the instructor input;
analyzing, via a large language model trained on the database, the first final grading data to generate a first summary;
receiving a plurality of lessons and importance rankings for each lesson from the instructor interface;
ordering the plurality of lessons in a prioritized sequence based on the importance rankings;
displaying the first final grading data, the first summary, and the plurality of lessons in the prioritized sequence to the user;
tracking completion of lessons in an activity log;
automatically prompting the user to submit a second video recording when a condition selected from a group consisting of completion of a predetermined number of lessons and elapse of a predetermined time period is satisfied;
receiving the second video recording depicting the physical movement at a second time point;
analyzing, via the image recognition model, the second video recording to generate a second suggested score;
receiving second final grading data from the instructor interface;
comparing the first final grading data to the second final grading data to generate a movement improvement metric;
generating score comparison data comparing the first final grading data and the second final grading data;
displaying the movement improvement metric and the score comparison data to the user;
enabling the user to share the first final grading data, the second final grading data, and the movement improvement metric to an external healthcare provider;
enabling the user to download the first final grading data, the second final grading data, the movement improvement metric, and historical assessment data as a local record; and
enabling the user to schedule an appointment with the instructor through the instructor interface.
